SEQUENCE LISTING (68 sequences)

SEQ ID NO: 1 - protein, 112 aa, Homo sapien IL-13
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 2 - protein, 111 aa, Murine IL-13
GPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 3 - protein, 111 aa, Porcine IL-13
GPVPPHSTALKELIEELVNITQNQKTPLCNGSMVWSVNLTTSMQYCAALESLINISDCSAIQKTQRMLSALCSHKPPSEQVPGKHIRDTKIEVAQFVKDLLKHLRMIFRHG

SEQ ID NO: 4 - protein, 112 aa, Bovine IL-13
SPVPSATALKELIEELVNITQNQKVPLCNGSMVWSLNLTSSMYCAALDSLISISNCSVIQRTKKMLNALCPHKPSAKQVSSEYVRDTKIEVAQFLKDLLRHSRIVFRNERFN

SEQ ID NO: 5 - protein, 111 aa, Canine IL-13
SPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 6 - protein, 111 aa, Rat IL-13
GPVRRSTSPPVALRELIEELSNITQDQKTSLCNSSMVWSVDLTAGGFCAALESLTNISSCNAIHRTQRILNGLCNQKASDVASSPPDTKIEVAQFISKLLNYSKQLFRYGH

SEQ ID NO: 7 - protein, 111 aa, Cynomolgus il-13
SPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLHLKKLFREGQFN

SEQ ID NO: 8 - protein, 112 aa, Rhesus IL-13
SPVPRSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGRFN

SEQ ID NO: 9 - protein, 112 aa, Marmoset IL-13
GPVPPYTALKELIEELVNITQNQKAPLCNGSMVWSINMTAGVYCAALESLINVSGCSAIEKTQRMLSGFCPHKVSAGQFSSLLVRDTKIEVAQFVKDLLRHLRKLFHQGTFN

SEQ ID NO: 10 - protein, 112 aa, Artificial Sequence
  Chimaeric Homo Sapien IL-13
GPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 11 - protein, 121 aa, Artificial Sequence
  Chimaeric Homo Sapien IL-13
GPVPPSTALRELIEELVNITQNQKAPLCNGSMVWSINLTAGMYCAALESLINVSGCSAIEKTQRMLGGFCPHKFNNFTVSFWLRVPKVSASHLEDTKIEVAQFVKDLLLHLKKLFREGRFN

SEQ ID NO: 12 - protein, 133 aa, Artificial Sequence
  Chimaeric Homo Sapien IL-13
FNNFTVSFWLRVPKVSASHLEGPVPPSTALRELIEELVNITQNQKAPLCNGSMVWSINLTAGMYCAALESLINVSGCSAIEKTQRMLGGFCPHKVSAGQFSSLHVRDTKIEVAQFVKDLLLHLKKLFREGRFN

SEQ ID NO: 13 - protein, 123 aa, Artificial Sequence
  Chimaeric Murine IL-13
GPVPRSVSLPLTLKELIEELSNITQDQTPLCNGSMVWSVDLAAGGFCVALDSLTNISNCNAIYRTQRILHGLCNRKFNNFTVSFWLRVPKVSASHLEDTKIEVAHFITKLLSYTKQLFRHGPF

SEQ ID NO: 14 - protein, 132 aa, Artificial Sequence
  Chimaeric Murine IL-13
FNNFTVSFWLRVPKVSASHLEGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 15 - protein, 132 aa, Artificial Sequence
  Chimaeric Murine IL-13
FNNFTVSFWLRVPKVSASHLEGPVPRSVSLPVTLKELIEELTNITQDQTPLCNGSMVWSVDLAAGGFCVALDSLTNISNCNAIFRTQRILHALCNRKAPTTVSSLPDTKIEVAHFITKLLTYTKNLFRRGPF

SEQ ID NO: 16 - protein, 249 aa, Artificial Sequence
  Chimaeric Homo Sapien IL-13
YVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 17 - protein, 220 aa, Artificial Sequence
  Chimaeric Homo Sapien IL-13
SSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 18 - protein, 133 aa, Artificial Sequence
  Chimaeric Homo Sapien IL-13
FNNFTVSFWLRVPKVSASHLEGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 19 - protein, 133 aa, Artificial Sequence
  Chimaeric Homo Sapien IL-13
FNNFTVSFWLRVPKVSASHLEGPVPPSSALKILIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 20 - protein, 112 aa, Artificial Sequence
  Chimaeric Homo Sapien IL-13
GPVPPSSALKELIEELANITQNQKAPLCNGSMVWSINLTAGMYCAALDSLINVSGCSAIERTQRILSAFCPHKVSAGQFSSLHVRDTKIEVAQFVTDLLVHLKRLFRQGRFN

SEQ ID NO: 21 - protein, 112 aa, Artificial Sequence
  Chimaeric Homo Sapien IL-13
GPVPPSTALKELIEELVNITQNQKAPLCNGSMVWSINLTAGMYCAALDSLINVSGCSAIERTQRILSAFCPHKVSAGQFSSLRVRDTKIEVAQFVTDLLVHLKKLFRQGTFN

SEQ ID NO: 22 - protein, 112 aa, Artificial Sequence
  Chimaeric Homo Sapien IL-13
GPVPPSSALRELIEELANITQNQKAPLCNGSMVWSINLTAGMYCAALESLINVSGCSAIDKTQRMLSAFCPHKVSAGQFSSLHVRDTKIEVAQFVKDLLVHLKRLFRDGRFN

SEQ ID NO: 23 - DNA, 1260 bp, Artificial Sequence
  Plasmid pCDNmIL13CDFC
aaccgtcagatcgcctggagacgccatcgaattcggtaccgccaccatggcgctctgggtgactgcagtcctggctcttgcttgccttggtggtctcgccgccccagggccggtgccacgttctgtgtctctccctctgacccttaaggagcttattgaggagctgagcaacatcacacaagaccagactcccctgtgcaacggcagcatggtatggagtgtggacctggccgctggcgggttctgtgtagccctggattccctgaccaacatctccaattgcaatgccatctaccgtacccagcgtattttgcatggcctctgtaaccgcaagtttaataattttaccgttagcttttggttgcgtgttcctaaagtatctgctagtcatttagaagataccaaaatcgaagtagcccactttattacaaaactgctcagctacacaaagcaactgtttcgccacggccccttcctggaggtcctgttccagggaccaggatccgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtgtagatccgttaacggttaccaactacctagggatccgttaacggttaccaact

SEQ ID NO: 24 - DNA, 1260 bp, Artificial Sequence
  Plasmid pCDNmIL13p30FC
aaccgtcagatcgcctggagacgccatcgaattcggtaccgccaccatggcgctctgggtgactgcagtcctggctcttgcttgccttggtggtctcgccgccccatttaataattttaccgttagcttttggttgcgtgttcctaaagtatctgctagtcatttagaagggccggtgccacgttctgtgtctctccctctgacccttaaggagcttattgaggagctgagcaacatcacacaagaccagactcccctgtgcaacggcagcatggtatggagtgtggacctggccgctggcgggttctgtgtagccctggattccctgaccaacatctccaattgcaatgccatctaccgtacccagcgtattttgcatggcctctgtaaccgcaaggcccccactacggtctccagcctccccgataccaaaatcgaagtagcccactttattacaaaactgctcagctacacaaagcaactgtttcgccacggccccttcctggaggtcctgttcccaggaccaggatccgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtgtagatccgttaacggttaccaactac

SEQ ID NO: 25 - DNA, 72 bp, Artificial Sequence
  primer
aacctgtttcgccgcggccccttcctggaggtcctgttcggtggaccaggatccgagcccaaatcggccgac

SEQ ID NO: 26 - DNA, 1260 bp, Artificial Sequence
  Plasmid pCDNcIL13newFC
aaccgtcagatcgcctggagacgccatcgaattcggtaccgccaccatggcgctctgggtgactgcagtcctggctcttgcttgccttggtggtctcgccgccccatttaataattttaccgttagcttttggttgcgtgttcctaaagtatctgctagtcatttagaagggccggtgccacgttctgtgtctctccctgtgacccttaaggagcttattgaggagctgaccaacatcacacaagaccagactcccctgtgcaacggcagcatggtatggagtgtggacctggccgctggcgggttctgtgtagccctggattccctgaccaacatctccaattgcaatgccatcttccgtacccagcgtattttgcatgccctctgtaaccgcaaggcccccactacggtctccagcctccccgataccaaaatcgaagtagcccactttattacaaaactgctcacctacacaaagaacctgtttcgccgcggccccttcctggaggtcctgttccagggaccaggatccgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtgtagatccgttaacggttaccaactac

SEQ ID NO: 27 - DNA, 24 bp, Artificial Sequence
  primer
gtgtctctccctctgacccttagg

SEQ ID NO: 28 - DNA, 24 bp, Artificial Sequence
  primer
cagttgctttgtgtagctgagcag

SEQ ID NO: 29 - DNA, 1200 bp, Artificial Sequence
  Plasmid pCDNIL13oldFC
aaccgtcagatcgcctggagacgccatcgaattcggtaccgccaccatggcgctctgggtgactgcagtcctggctcttgcttgccttggtggtctcgccgccccagggccggtgccacgttctgtgtctctccctctgacccttagggagctcattgaggagctggtcaacatcacacaagaccagactcccctgtgcaacggcagcatggtatggagtgtggacctggccgctggcgggtactgtgcagccctggaatccctgaccaatatttccaattgcaatgccatcgagaagacccagaggatgctgggcggactctgtaaccgcaaggcccccactacggtctccagcctccccgataccaaaatcgaggtggcccagtttgtaaaggacctgctcagctacacaaagcaactgtttcgccacggccccttcctggaggtcctgttccagggaccaggatccgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtgtagatccgttaacggttaccaactaccta

SEQ ID NO: 30 - protein, 3 aa, Homo sapien
PVP

SEQ ID NO: 31 - protein, 6 aa, Homo sapien
ELIEEL

SEQ ID NO: 32 - protein, 4 aa, Homo sapien
NITQ

SEQ ID NO: 33 - protein, 3 aa, Homo sapien
LCN

SEQ ID NO: 34 - protein, 5 aa, Homo sapien
SMVWS

SEQ ID NO: 35 - protein, 2 aa, Homo sapien
SL

SEQ ID NO: 36 - protein, 2 aa, Homo sapien
AI

SEQ ID NO: 37 - protein, 2 aa, Homo sapien
TQ

SEQ ID NO: 38 - protein, 7 aa, Homo sapien
DTKIEVA

SEQ ID NO: 39 - protein, 2 aa, Homo sapien
LL

SEQ ID NO: 40 - protein, 2 aa, Homo sapien
LF

SEQ ID NO: 41 - protein, 16 aa, Homo sapien
PSTALRELIEELVNIT

SEQ ID NO: 42 - protein, 10 aa, Homo sapien
MYCAALESLI

SEQ ID NO: 43 - protein, 9 aa, Homo sapien
KTQRMLSGF

SEQ ID NO: 44 - protein, 17 aa, Homo sapien
AQFVKDLLLHLKKLFRE

SEQ ID NO: 45 - protein, 8 aa, Homo sapien
GPVPPSTA

SEQ ID NO: 46 - protein, 24 aa, Homo sapien
ITQNQKAPLCNGSMVWSINLTAGM

SEQ ID NO: 47 - protein, 7 aa, Homo sapien
INVSGCS

SEQ ID NO: 48 - protein, 19 aa, Homo sapien
FCPHKVSAGQFSSLHVRDT

SEQ ID NO: 49 - protein, 13 aa, Homo sapien
LHLKKLFREGRFN

SEQ ID NO: 50 - protein, 14 aa, Homo sapien
QYIKANSKFIGITE

SEQ ID NO: 51 - protein, 21 aa, Homo sapien
FNNFTVSFWLRVPKVSASHLE

SEQ ID NO: 52 - protein, 6 aa, Homo sapien
FNNFTV

SEQ ID NO: 53 - protein, 21 aa, Homo sapien
DIEKKIAKMEKASSVFNVVNS

SEQ ID NO: 54 - protein, 15 aa, Homo sapien
LSEIKGVIVHRLEGV

SEQ ID NO: 55 - protein, 15 aa, Homo sapien
FFLLTRILTIPQSLD

SEQ ID NO: 56 - protein, 19 aa, Homo sapien
PVFAGANYAAWAVNVAQVI

SEQ ID NO: 57 - protein, 20 aa, Homo sapien
VHHNTEEIVAQSIALSSLMV

SEQ ID NO: 58 - protein, 20 aa, Homo sapien
QSIALSSLMVAQAIPLVGEL

SEQ ID NO: 59 - protein, 20 aa, Homo sapien
VDIGFAAYNFVESIINLFQV

SEQ ID NO: 60 - protein, 20 aa, Homo sapien
QGESGHDIKITAENTPLPIA

SEQ ID NO: 61 - protein, 20 aa, Homo sapien
GVLLPTIPGKLDVNKSKTHI

SEQ ID NO: 62 - DNA, 20 bp, Homo sapien
tccatgacgttcctgacgtt

SEQ ID NO: 63 - DNA, 18 bp, Homo sapien
tctcccagcgtgcgccat

SEQ ID NO: 64 - DNA, 30 bp, Homo sapien
accgatgacgtcgccggtgacggcaccacg

SEQ ID NO: 65 - DNA, 24 bp, Homo sapien
tcgtcgttttgtcgttttgtcgtt

SEQ ID NO: 66 - DNA, 20 bp, Homo sapien
tccatgacgttcctgatgct

SEQ ID NO: 67 - DNA, 71 bp, Homo sapien
caactgtttcgccacggccccttcctggaggtcctgttcggtggaccaggatccgagcccaatcggccgac

SEQ ID NO: 68 - DNA, 25 bp, Homo sapien
ctaggtagttggtaaccgttaacgg

(19) United States
(12) Patent Application Publication
(10) Pub. No.: US 2006/0147417 A1
(43) Pub. Date: Jul. 6, 2006

(54) Immunogenic composition comprising an il-13 element and t cell epitopes, and its therapeutic use

(76) Inventors: Claire Ashman, Stevenage, Hertfordshire (GB); Jonathan Henry Ellis, Stevenage, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/526,060

(22) PCT Filed: Aug. 28, 2003
(86) PCT No.: PCT/GB03/03703

§ 371 Date: Feb. 28, 2005

(30) Foreign Application Priority Data
Aug. 30, 2002 (GB) 0220212.5
Feb. 28, 2003 (GB) 0304672.9

(51) Int. Cl. A61K 38/20; A61K 39/02

(52) U.S. Cl. 424/85.2; 424/190.1

(57) ABSTRACT

The present invention relates to isolated immunogens and their use in the treatment of diseases that are treatable with neutralization of IL-13, such as COPD, asthma and atopic disorders such as hayfever, contact allergies and atopic dermatitis. In particular the invention relates to the neutralization of the biological effects of IL-13 by raising an immune response against the IL-13 by vaccination of a mammal with immunogens comprising the native or mutated amino acid sequence of IL-13, and foreign T-helper epitopes either inserted in, or attached to the IL-13 sequence or present in carrier polypeptides. Also provided by the present invention are DNA vaccines that comprise a polynucleotide sequence that encodes the immunogens of the present invention. The invention further relates to pharmaceutical compositions comprising such immunogens and their use in medicine and to methods for their production.

The present invention relates to isolated immunogens and their use in the treatment of diseases that are treatable with neutralisation of IL-13, such as COPD, asthma and atopic disorders such as hayfever, contact allergies and atopic dermatitis. Most preferably the immunogens are used in the treatment of asthma. In particular the invention relates to the neutralisation of the biological effects of IL-13 by raising an immune response against the IL-13 by vaccination of a mammal with immunogens comprising the native or mutated amino acid sequence of IL-13, and foreign T-helper epitopes either inserted in, or attached to the IL-13 sequence or present in carrier polypeptides. Also provided by the present invention are DNA vaccines that comprise a polynucleotide sequence that encodes the immunogens of the present invention. The invention further relates to pharmaceutical compositions comprising such immunogens and their use in medicine and to methods for their production.

### BACKGROUND TO THE INVENTION

COPD is an umbrella term to describe diseases of the respiratory tract, which shows similar symptoms to asthma and is treated with the same drugs. COPD is characterised by a chronic, progressive and largely irreversible airflow obstruction. The contribution of the individual to the course of the disease is unknown, but smoking cigarettes is thought to cause 90% of the cases. Symptoms include coughing, chronic bronchitis, breathlessness and respiratory injections. Ultimately the disease will lead to severe disability and death.

Asthma is a chronic lung disease, caused by inflammation of the lower airways and is characterised by recurrent breathing problems. Airways of patients are sensitive and swollen or inflamed to some degree all the time, even when there are no symptoms. Inflammation results in narrowing of the airways and reduces the flow of air in and out of the lungs, making breathing difficult and leading to wheezing, chest tightness and coughing. Asthma is triggered by super-sensitivity towards allergens (e.g. dust mites, pollens, moulds), irritants (e.g. smoke, fumes, strong odours), respiratory infections, exercise and dry weather. The triggers irritate the airways and the lining of the airways swell to become even more inflamed, mucus then clogs up the airways and the muscles around the airways tighten up until breathing becomes difficult and stressful and asthma symptoms appear.

Atopic disorders refers to a group of diseases that are hereditary and often occur together, including asthma, allergies such as hay fever, and atopic dermatitis. Atopic dermatitis is a chronic disease that affects the skin. In atopic dermatitis, the skin becomes extremely itchy and inflamed, causing redness, swelling, cracking, weeping, crusting, and scaling. Atopic dermatitis most often affects infants and young children, but it can continue into adulthood or first show up later in life. In most cases, there are periods of time when the disease is worse, called exacerbations or flares, followed by periods when the skin improves or clears up entirely, called remissions. Many children with atopic dermatitis will experience a permanent remission of the disease when they get older, although their skin often remains dry and easily irritated. Environmental factors can bring on symptoms of atopic dermatitis at any time in the lives of individuals who have inherited the atopic disease trait. Atopic dermatitis is often referred to as “eczema,” which is a general term for the many types of dermatitis. Atopic dermatitis is the most common of the many types of eczema. Several have very similar symptoms.

The way the skin is affected by atopic dermatitis can be changed by patterns of scratching and resulting skin infections. Some people with the disease develop red, scaling skin where the immune system in the skin is becoming very activated. Others develop thick and leathery skin as a result of constant scratching and rubbing. This condition is called lichenification. Still others develop papules, or small raised bumps, on their skin. When the papules are scratched, they may open (excoriations) and become crusty and infected.

Many factors or conditions can make symptoms of atopic dermatitis worse, further triggering the already overactive immune system in the skin, aggravating the itch-scratch cycle, and increasing damage to the skin. These exacerbating factors can be broken down into two main categories: irritants (such as wool or synthetic fibers, rough or poorly fitting clothing, soaps and detergents, some perfumes and cosmetics, chlorine, mineral oil, some solvents, dust or sand) and allergens (such as pollen, dog or cat dander, and dust mite allergens). Emotional factors and some infections can also influence atopic dermatitis.

If a flare of atopic dermatitis does occur, several methods can be used to treat the symptoms. Corticosteroids as topical creams are the most frequently used treatment, although systemic administration is also used in some severe cases. Sometimes over-the-counter preparations are used, but in many cases the doctor will prescribe a stronger corticosteroid cream or ointment. An example of a commonly prescribed corticosteroid is prednisone. Side effects of repeated or long-term use of topical corticosteroids can include thinning of the skin, infections, growth suppression (in children), and stretch marks on the skin. Antibiotics to treat skin infections may be applied directly to the skin in an ointment, but are usually more effective when taken by mouth. Phototherapy (treatment with light) that uses ultraviolet A or B light waves, or both together, can be an effective treatment for mild to moderate dermatitis in older children (over 12 years old) and adults. In adults, immunosuppressive drugs, such as cyclosporine, are also used to treat severe cases of atopic dermatitis that have failed to respond to any other forms of therapy. The side effects of cyclosporine can include high blood pressure, nausea, vomiting, kidney problems, headaches, tingling or numbness, and a possible increased risk of cancer and infections.

Because of the unmet medical need therefor and the side affects of existing therapies there is a need for alternative treatments for atopic diseases in general, and in particular for treatments for asthma and atopic dermatitis.

IL-13 is a Th2-type cytokine that is closely related to IL-4. A number of recent papers have defined the role for IL-13 in driving pathology in the ovalbumin model of allergenic asthma (Wills-Karp et al, 1998, Science:282:2258-2261; Grunig et al, 1998, Science 282:2261-2263). In this work, mice previously sensitised to ovalbumin were injected with a soluble IL-13 receptor which binds and neutralises IL-13. Airway hyper-responsiveness to acetylcholine challenge was reduced in the treated group. Histological analysis revealed that treated mice had reversed the goblet-cell metaplasia seen in controls. In complementary experiments, lung IL-13 levels were raised by over-expression in a transgenic mouse or by installation of protein into the trachea in wild-type mice. In both settings, airway hyper-responsiveness, eosinophil invasion and increased mucus production were seen (Zhu et al, 1999, J. Clin. Invest. 103:779-788).

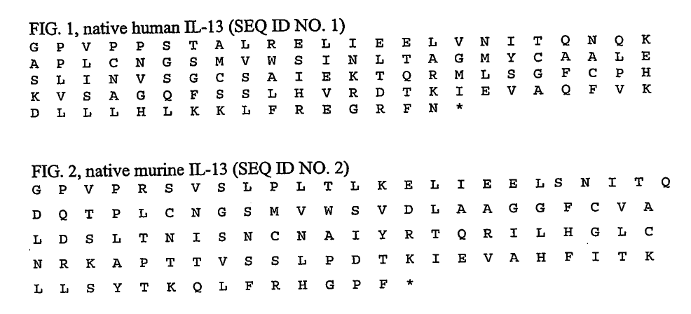

The sequence of the mature form of human IL-13 is provided in SEQ ID No. 1 and is shown in FIG. 1.

The sequence of the mature form of murine IL-13 is provided in SEQ ID No. 2 and is shown in FIG. 2.

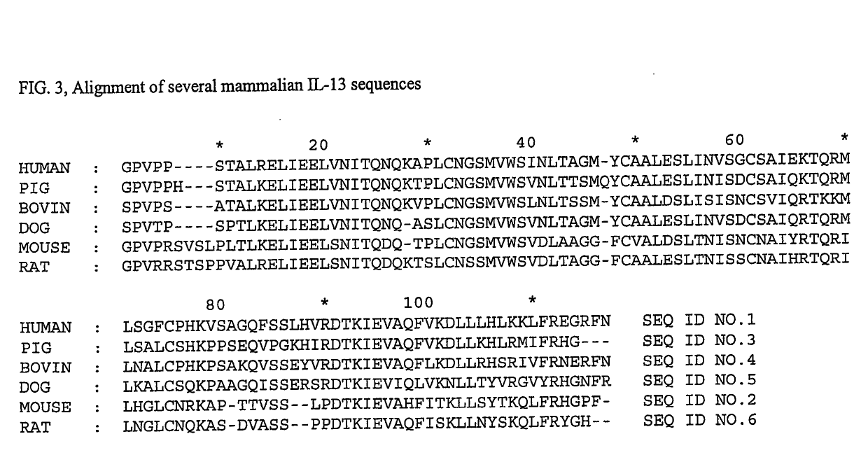

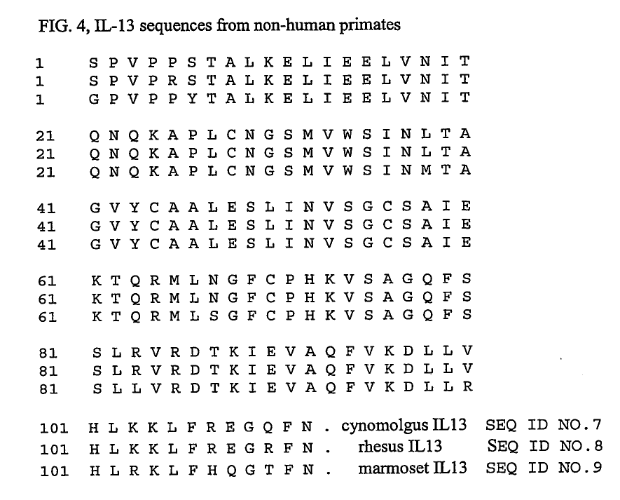

Sequences for IL-13 from several mammalian species and non-human primates are shown in FIG. 3 and FIG. 4 (SEQ ID NO.s 3 to 9)

As a result of the various problems associated with the production, administration and tolerance of monoclonal antibodies there is an increased focus on methods of instructing the patient's own immune system to generate endogenous antibodies of the appropriate specificity by means of vaccination. However, mammals do not generally have high-titre antibodies against self-proteins present in serum, as the immune system contains homeostatic mechanisms to prevent their formation. The importance of these “tolerance” mechanisms is illustrated by diseases like myasthenia gravis, in which auto-antibodies directed to the nicotinic acetylcholine receptor of skeletal muscle cause weakness and fatigue (Drachman, 1994, N Engl J Med 330:1797-1810).

A number of techniques have been designed with the aim of breaking “tolerance” to self antigen. One technique involves chemically cross-linking the self-protein (or peptides derived from it) to a highly immunogenic carrier protein, such as keyhole limpet haemocyanin (“Antibodies: A laboratory manual” Harlow, E and Lane D. 1988. Cold Spring Harbor Press).

A variant on the carrier protein technique involves the construction of a gene encoding a fusion protein comprising both carrier protein (for example hepatitis B core protein) and self-protein (The core antigen of hepatitis B virus as a carrier for immunogenic peptides”, Biological Chemistry. 380(3):277-83, 1999). The fusion gene may be administered directly as part of a nucleic acid vaccine. Alternatively, it may be expressed in a suitable host cell in vitro, the gene product purified and then delivered as a conventional vaccine, with or without an adjuvant.

Another approach has been described by Dalum and colleagues wherein a single class II MHC-restricted epitope is inserted into the target molecule. They demonstrated the use of this method to induce antibodies to ubiquitin (Dalum et al, 1996, J Immunol 157:4796-4804; Dalum et al, 1997, Mol Immunol 34:1113-1120) and the cytokine TNF (Dalum et al, 1999, Nature Biotech 17:666-669). As a result, all T cell help must arise either from this single epitope or from junctional sequences. Such an approach is also described in EP 0 752 886 B1, WO 95/05849, and WO 00/65058.

Treatment therapies, some including vaccination, for the neutralisation of several cytokines are known. WO 00/65058 describes a method of down regulating the function of the cytokine IL-5, and its use in the treatment of asthma. In this study, the IL-5 sequence was modified by a number of techniques to render it immunogenic, amongst which there is described an IL-5 immunogen supplemented with foreign T-cell epitopes, whilst maintaining the IL-5 B cell epitopes. WO 01/62287 discloses IL-13, amongst a long list of potential antigens, for use in allergy or asthma vaccines. WO 00/06937 discloses cytokine derivatives that are functionally inactivated for use as vaccine antigens. Chimaeric IL-13 immunogens are disclosed in the co-pending patent application WO 02/070711.

There remains a need to provide improved immunotherapeutic treatments for asthma, and improved immunogens for raising neutralising anti-IL-13 immune responses.

### SUMMARY OF THE INVENTION

The present invention provides pharmaceutical compositions comprising modified “self” IL-13 immunogens, wherein the IL-13 immunogen is modified to include foreign T-cell helper epitopes. The pharmaceutical composition is preferably for use in human therapy, and in this composition the IL-13 sequence is a human sequence or other sequence that is capable of generating an immune response that recognises human IL-13; and the T-cell helper epitopes are “foreign” with respect to human self-proteins. Preferably the T-helper epitopes are also foreign with respect to other IL-13 sequences from other species. However, animal pharmaceutical products are not excluded, for example canine or other veterinary species pharmaceutical products can be made in an analogous fashion to that described for human vaccines above.

The compositions of the present invention comprise an IL-13 element and an additional element for providing T-cell help.

IL-13 Element

The IL-13 element, in its broadest form, is any sequence that is capable of driving an immune response that recognises and neutralises the biological effects of IL-13. Preferably, the IL-13 is human IL-13.

In this context of the present invention the entire IL-13 sequences may be used, or functional equivalent fragments thereof. Accordingly, references in this text to IL-13 sequences may encompass the entire sequence or fragments or truncates thereof.

The IL-13 element may comprise the native IL-13 sequence or a mutated form thereof. Accordingly, the IL-13 sequence may be, for example, native human IL-13 or fragment thereof.

In an alternative embodiment of the present invention the immunogens comprise a chimaeric IL-13 sequence that comprise substitution mutations to swap one or more of the human sequence amino acids with the equivalent amino acids found in the same positions within the sequence of IL-13 from another mammalian species. In the context of a human vaccine immunogen, the object of the chimaeric sequences is to maximise the amino acid sequence diversity between the immunogen and human native IL-13, whilst keeping maximal shape and conformational homology between the two compositions. The chimaeric immunogen achieves this by substituting amino acids found in regions predicted to be masked from the surface. Most preferably the amino acids are substituted with amino acids that are found in equivalent positions within an IL-13 sequence from another mammalian species. In this way, sequence diversity is achieved with minimal alteration to the overall shape/configuration of the immunogen.

In one aspect of the present invention, there is provided a human IL-13 immunogen that comprises substitution mutations in areas that are associated with alpha helical regions, which substitutions involve swapping the human amino acid with the amino acid that appears in the same position within the IL-13 sequence of a different mammalian species.

Most preferably, there are substitution mutations in a plurality of sites within the IL-13 sequence, wherein at least two or more of the mutation sites comprise a substitution involving amino acids taken from different non-human mammalian species, more preferably the substitutions involve amino acids taken from 3 or more different non-human mammalian species, and most preferably the substitutions involve amino acids taken from 4 or more different non-human mammalian species.

Preferably, the substitutions do not occur in at least six of the areas of high interspecies conservation: 3PVP (SEQ ID NO. 30), 12ELIEEL (SEQ ID NO. 31), 19NITQ (SEQ ID NO. 32), 28LCN (SEQ ID NO. 33), 32SMVWS (SEQ ID NO. 34), 50SL (SEQ ID NO. 35), 60AI (SEQ ID NO. 36), 64TQ (SEQ ID NO. 37), 87DTKIEVA (SEQ ID NO. 38), 99LL (SEQ ID NO. 39), 106LF (SEQ ID NO. 40).

The preferred IL-13 element of the immunogens of the present invention are human chimaeric IL-13 sequences which have a similar conformational shape to native human IL-13 whilst having sufficient amino acid sequence diversity to enhance its immunogenicity when administered to a human, characterised in that the chimaeric IL-13 immunogen has the sequence of human IL-13 comprising:

(a) substitution mutations in at least two of the following alpha helical regions: PSTALRELIEELVNIT (SEQ ID NO. 41), MYCAALESLI (SEQ ID NO. 42), KTQRMLSGF (SEQ ID NO. 43) or AQFVKDLLLHLKKLFRE (SEQ ID NO. 44),

(b) comprises in unmutated form at least six of the following regions of high inter-species conservation 3PVP (SEQ ID NO. 30), 12ELIEEL (SEQ ID NO. 31), 19NITQ (SEQ ID NO. 32), 28LCN (SEQ ID NO. 33), 32SMVWS (SEQ ID NO. 34), 50SL (SEQ ID NO. 35), 60AI (SEQ ID NO. 36), 64TQ (SEQ ID NO. 37), 87DTKIEVA (SEQ ID NO. 38), 99LL (SEQ ID NO. 39), 106LF (SEQ ID NO. 40), and

(c) optionally comprises a mutation in any of the remaining amino acids,

wherein any substitution performed in steps a, b or c is a structurally conservative substitution.

The numerical prefix to the amino acids listed, refers to the positional number of the amino acid sequence in the mature form of human IL-13, wherein the first residue “G” is assigned the number 2.

In the context of step (a) of the above chimaeric IL-13 element, preferably at least two, more preferably at least three and most preferably all four alpha helical regions comprise at least one substitution mutation. In the context of step (b) preferably at least 7, more preferably at least 8, more preferably at least 9, more preferably at least 10, and most preferably all 11 of the regions are unmutated.

Preferably greater than 50% of these substitutions or mutations in the above chimaeric IL-13 element, comprise amino acids taken from equivalent positions within the IL-13 sequence of a non-human. More preferably more than 60, or 70, or 80 percent of the substitutions comprise amino acids taken from equivalent positions within the IL-13 sequence of a non-human mammal. Most preferably, each substitution or mutation comprise amino acids taken from equivalent positions within the IL-13 sequence of a non-human mammal.

Again in the context of the chimaeric human IL-13 element, preferably greater than 50% of these substitutions or mutations occur in regions of human IL-13 which are predicted to be alpha helical in configuration. More preferably more than 60, or 70, or 80 percent of the substitutions or mutations occur in regions of human IL-13 which are predicted to be alpha helical in configuration. Most preferably, each substitution or mutation occurs in regions of human IL-13 which are predicted to be alpha helical in configuration.

Again in the context of the chimaeric human IL-13 elements, preferably the human IL-13 sequence comprises between 2 and 20 substitutions, more preferably between 6 and 15 substitutions and most preferably 13 substitutions.

In the case of a human IL-13 vaccine, the IL-13 immunogen could be based on an orthologous IL-13 sequence (such as the murine IL-13 sequence) wherein the murine B-cell epitopes (surface exposed regions) are substituted for the equivalent human sequences. In this embodiment the murine “backbone” will provide foreign T-cell epitopes, in addition to the supplemental promiscuous T-cell epitopes (such as P2 or P30) which are added either at the termini or within the chimaera sequence.

A preferred chimaeric human IL-13 immunogen comprises the sequence of human IL-13, wherein the amino acid sequence comprises conservative substitutions, or substitutions characteristic of amino acids present at equivalent positions within the IL-13 sequence of a non-human species, present in at least six of the following 13 positions 8T, 11R, 18V, 49E, 62K, 66M, 69G, 84H, 97K, 101L, 105K, 109E, 111R. Most preferably such a chimaeric human IL-13 immunogen comprises at least 6, and preferrably all, of the following substitutions:

| Position | Substitution | Species |
|---|---|---|
| 8 | T->S | Synthetic |
| 11 | R->K | pig, cow, dog, mouse, gerbil, |
| | | cyno, rhesus, marmoset. |
| 18 | V->A | Synthetic |
| 49 | E->D | cow, mouse, gerbil. |
| 62 | K->R | cow, dog, mouse, rat. |
| 66 | M->I | Mouse, gerbil, rat. |
| 69 | G->A | Cow, pig, dog |
| 84 | H->R | Dog, rhesus, cyno |
| 97 | K->T | Mouse |
| 101 | L->V | Cyno, rhesus |
| 105 | K->R | Synthetic |
| 109 | E->Q | Marmoset |
| 111 | R->T | Marmoset |

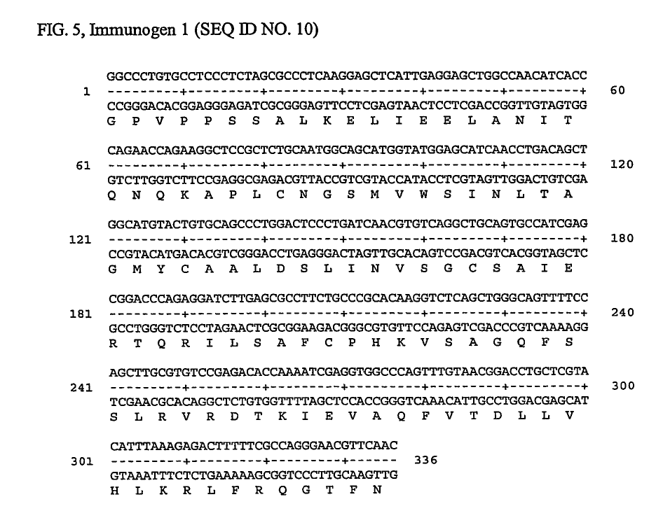

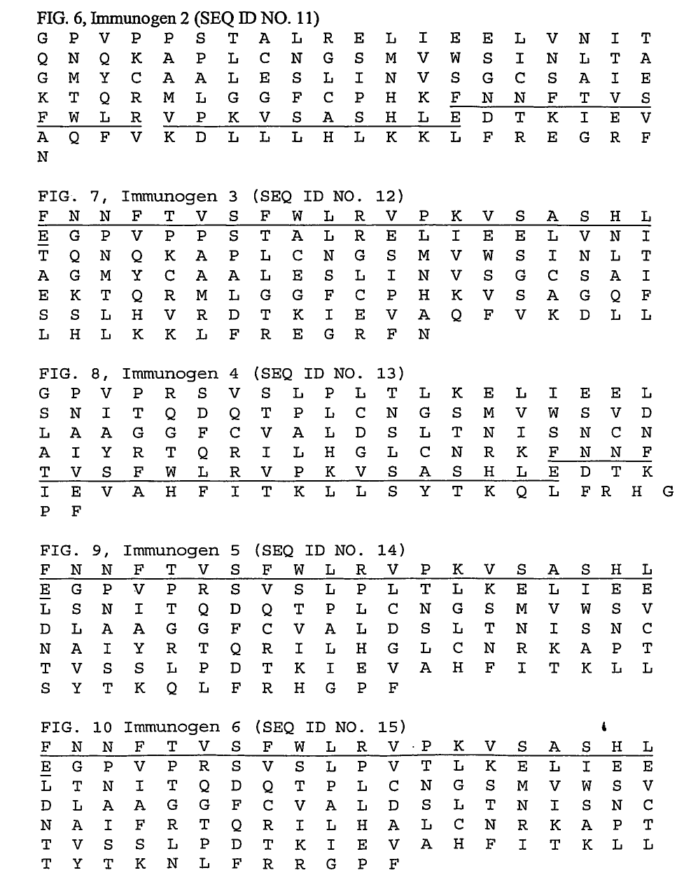

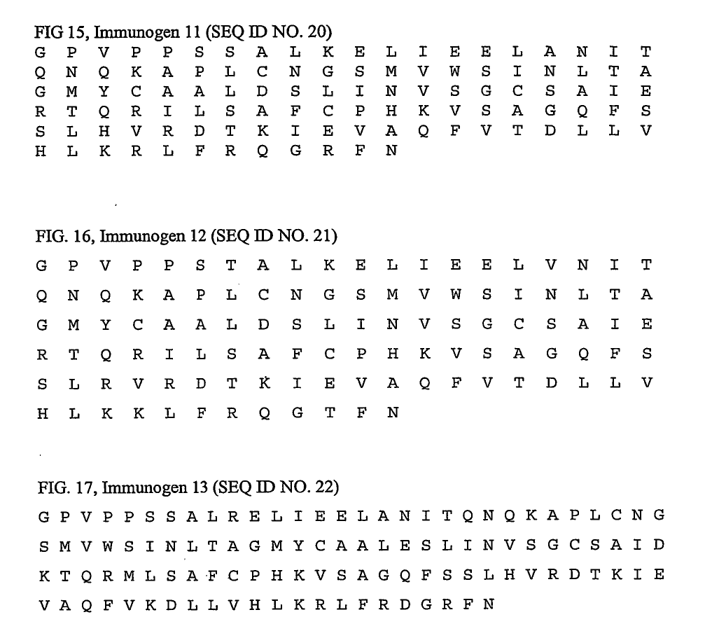

The chimaeric IL-13 that comprises each of these listed substitutions is a preferred IL-13 element (Immunogen 1, SEQ ID NO. 10) and is shown in FIG. 5. Other highly preferred IL-13 elements are Immunogen 11 (SEQ ID NO. 20, see FIG. 15), Immunogen 12 (SEQ ID NO. 21, see FIG. 16) and Immunogen 13 (SEQ ID NO. 22, see FIG. 17).

The IL-13 element may also optionally further comprise a mutation that abolishes the biological activity of the immunogen. The following substitutions can be used to inactivate human IL13 bioactivity: E 12 to I, S, or Y; E12 to K; R 65 to D; S 68 to D; R 108 to D.

In certain aspects of the present invention immunogenic fragments of the native IL-13 sequence may be used, for example in the presentation of immunogenic peptides in Hepatitis B core particles or in the context of chimaeric immunogens described above. In these contexts immunogenic fragments of the human IL-13 sequences preferably contain the B-cell epitopes in the human IL-13 sequence, and preferably at least one or more of the following short sequences:

| | |
|---|---|
| GPVPPSTA | (SEQ ID NO. 45) |
| ITQNQKAPLCNGSMVWSINLTAGM | (SEQ ID NO. 46) |
| INVSGCS | (SEQ ID NO. 47) |
| FCPHKVSAGQFSSLHVRDT | (SEQ ID NO. 48) |
| LHLKKLFREGRFN | (SEQ ID NO. 49) |

The polypeptide of the invention may be further modified by mutation, for example substitution, insertion or deletion of amino-acids in order to add desirable properties (such as the addition of a sequence tag that facilitates purification or increase immunogenicity) or remove undesirable properties (such as an unwanted agonistic activity at a receptor) or transmembrane domains. In particular the present invention specifically contemplates fusion partners that ease purification such as poly histidine tags or GST expression partners that enhance expression. A preferred tag or expression partner is immunoglobulin FC of human IgG1 fused to the C-terminus of the IL-13 molecule.

Other mutations, outside of those regions that are to be left unmutated due to their high level of conservation between species, may occur in the IL-13 sequence. Preferably such mutations are conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5), asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. These are preferred conservative substitutions.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

Element to Provide T-Cell Help.

Associated with the IL-13 element to make the immunogens of the present invention, are elements that provide foreign T-cell help. Most preferably the T-cell helper epitopes are foreign to human sequences, but also foreign with respect to any IL-13 sequences from non-human mammals. Preferably the T-cell helper epitopes used are small and are added to the IL-13 sequence by an addition or substitution event within, or at the terminal ends of, the IL-13 sequence by synthetic, recombinant or molecular biological means. Alternatively the T-cell helper epitopes may be added via chemical coupling of the IL-13 polypeptide to a carrier protein comprising the T-cell helper epitopes. The IL-13 sequences, or functionally equivalent fragments thereof, may also be associated with the T-cell helper epitopes in a fusion protein, wherein the two are recombinantly manufactured together, for example a Hepatitis B core protein incorporating IL-13 sequences.

In the aspects of the present invention where small T-cell helper epitopes are used, a “foreign T-cell helper epitope” or “T-cell epitope” is a peptide which is able to bind to an MHC II molecule and stimulates T-cells in an animal species. Preferred foreign T-cell epitopes are promiscuous epitopes, ie. epitopes that bind multiple different MHC class II molecules in an animal species or population (Panina-Bordignon et al, Eur. J. Immunol. 1989, 19:2237-2242; Reece et al, J. Immunol. 1993, 151:6175-6184; WO 95/07707).

In order for the immunogens of the present invention to be clinically effective in a complex outbred human population, it may be advantageous to include several foreign T-cell epitopes. Promiscuous epitopes may also be another way of achieving this same effect, including naturally occurring human T-cell epitopes such as those from tetanus toxoid (e.g. the P2 and P30 epitopes, diphtheria toxoid, influenza virus haemagluttinin (HA), and P. falciparum CS antigen. The most preferred T-cell epitopes for use in the present invention are P2 and P30 from tetanus toxoid

A number of promiscuous T-cell epitopes have been described in the literature, including: WO 98/23635; Southwood et al., 1998, J. Immunol., 160: 3363-3373; Sinigaglia et al., 1988, Nature, 336: 778-780; Rammensee et al., 1995, Immunogenetics, 41: 4, 178-228; Chicz et al., 1993, J. Exp. Med., 178:27-47; Hammer et al., 1993, Cell 74:197-203; and Falk et al., 1994, Immunogenetics, 39: 230-242. The promiscuous T-cell epitope can also be an artificial sequence such as “PADRE” (WO 95/07707).

The heterologous T-cell epitope is preferably selected from the group of epitopes that will bind to a number of individuals expressing more than one MHC II molecules in humans. For example, epitopes that are specifically contemplated are P2 and P30 epitopes from tetanus toxoid, Panina-Bordignon Eur. J. Immunol 19 (12), 2237 (1989). In a preferred embodiment the heterologous T-cell epitope is P2 or P30 from Tetanus toxin.

The P2 epitope has the sequence QYIKANSKFIGITE (SEQ ID NO. 50) and corresponds to amino acids 830-843 of the Tetanus toxin.

The P30 epitope (residues 947-967 of Tetanus Toxin) has the sequence FNNFTVSFWLRVPKVSASHLE (SEQ ID NO. 51). The FNNFTV (SEQ ID NO. 52) sequence may optionally be deleted. Other universal T epitopes can be derived from the circumsporozoite protein from Plasmodium falciparum—in particular the region 378-398 having the sequence DIEKKIAKMEKASSVFNVVNS (SEQ ID NO. 53; Alexander J, (1994) Immunity 1 (9), p 751-761).

Another epitope is derived from Measles virus fusion protein at residue 288-302 having the sequence LSEIKGVIVHRLEGV (SEQ ID NO. 54; Partidos C D, 1990, J. Gen. Virol 71(9) 2099-2105).

Yet another epitope is derived from hepatitis B virus surface antigen, in particular amino acids, having the sequence FFLLTRILTIPQSLD (SEQ ID NO. 55).

Another set of epitopes is derived from diphteria toxin. Four of these peptides (amino acids 271-290, 321-340, 331-350,351-370) map within the T domain of fragment B of the toxin, and the remaining 2 map in the R domain (411-430, 431-450):

| | |
|---|---|
| PVFAGANYAAWAVNVAQVI | (SEQ ID NO. 56) |
| VHHNTEEIVAQSIALSSLMV | (SEQ ID NO. 57) |
| QSIALSSLMVAQAIPLVGEL | (SEQ ID NO. 58) |
| VDIGFAAYNFVESII NLFQV | (SEQ ID NO. 59) |
| QGESGHDIKITAENTPLPIA | (SEQ ID NO. 60) |
| GVLLPTIPGKLDVNKSKTHI | (SEQ ID NO. 61) |

(Raju R., Navaneetham D., Okita D., Diethelm-Okita B., McCormick D., Conti-Fine B. M. (1995) Eur. J. Immunol. 25: 3207-14.)

A particularly preferred element to provide T-cell help, is a fusion partner called “CPC” (clyta-P2-clyta) which is disclosed in PCT/EP03/06096.

Most preferably the foreign T-cell helper epitopes are “foreign” in that they are not tolerated by the host immune system, and also in that they are not sequences that are derived or selected from any IL-13 sequence from another species (non-vaccinee).

In the aspect of the present invention where native self IL-13 is coupled to a T-helper epitope bearing immunogenic carrier, the conjugation can be carried out in a manner well known in the art. Thus, for example, for direct covalent coupling it is possible to utilise a carbodiimide, glutaraldehyde or (N-[γ-maleimidobutyryloxy]succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

The types of carriers used in the immunogens of the present invention will be readily known to the man skilled in the art. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD). Alternatively the IL-13 may be directly conjugated to liposome carriers, which may additionally comprise immunogens capable of providing T-cell help. Preferably the ratio of IL-13 to carrier molecules is in the order of 1:1 to 20:1, and preferably each carrier should carry between 3-15 IL-13 molecules.

In an embodiment of the invention a preferred carrier is Protein D from Haemophilus influenzae (EP 0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D ⅓^{rd} (comprising the N-terminal 100-110 amino acids of protein D (GB 9717953.5)).

Another preferred method of presenting the IL-13, or immunogenic fragments thereof, is in the context of a recombinant fusion molecule. For example, EP 0 421 635 B describes the use of chimaeric hepadnavirus core antigen particles to present foreign peptide sequences in a virus-like particle. As such, immunogens of the present invention may comprise IL-13 presented in chimaeric particles consisting of hepatitis B core antigen. Additionally, the recombinant fusion proteins may comprise IL-13 and a carrier protein, such as NS1 of the influenza virus. For any recombinantly expressed protein which forms part of the present invention, the nucleic acid which encodes said immunogen also forms an aspect of the present invention.

Preferred Immunogens

In the sections above, preferred definitions of the IL-13 element and the element to provide T-cell help have been described. For the compositions of the present invention, it is intended that this document discloses each individual preferred element from the IL-13 element section in combination with each individual preferred element from the element to provide T-cell help section. Particularly preferred are combinations of Immunogens 1, 11, 12 or 13, and a carrier protein or promiscuous T-cell helper epitope. Preferred carrier protein or promiscuous T-cell helper epitopes include Protein D, CPC, P2 or P30.

Specifically disclosed preferred combinations of elements to form preferred immunogens are listed herebelow.

When the IL-13 element is native human IL-13, and the element that provides T-cell help is a promiscuous T-cell epitope, preferred examples include: Immunogen 2 (see FIG. 6, SEQ ID NO. 11), which comprises human IL-13 with P30 inserted (underlined) into the protein (substituted for the looped region between alpha helices C and D of human IL13).

Immunogen 3 (FIG. 7, SEQ ID NO. 12) is a Human IL-13 immunogen with N-terminal P30.

Immunogen 4 (FIG. 8, SEQ ID NO. 13) is a murine IL-13 with p30 inserted into the protein (substituted for the looped region between alpha helices C and D of mouse IL13) this is an example of a mouse version of an IL13 autovaccine. The p30 region is underlined.

Immunogen 5 (FIG. 9, SEQ ID NO. 14) is a murine IL13 with p30 at the N-terminus. This is an example of a mouse version of an IL13 autovaccine. The p30 region is underlined and is positioned at the N-terminus of the mature mouse IL13 protein sequence.

Specific examples where the IL-13 element is provided as a chimaeric IL-13 immunogen include:

Immunogen 6 (FIG. 10, SEQ ID NO. 15). This is an example of a mouse version of this form of the vaccine, where there is “human backbone” sequence grafted to murine B-cell surface exposed epitopes, with P30 added at the N-terminus.

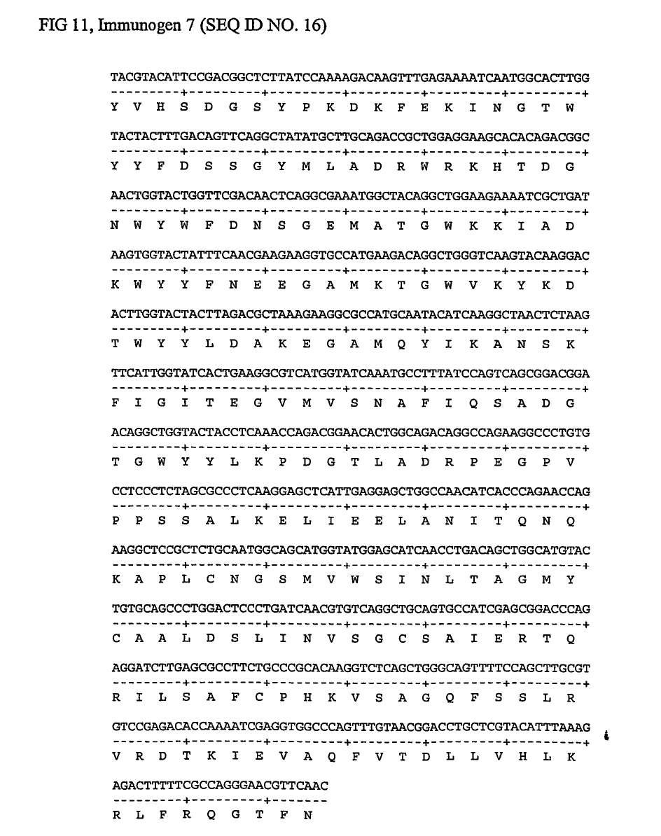

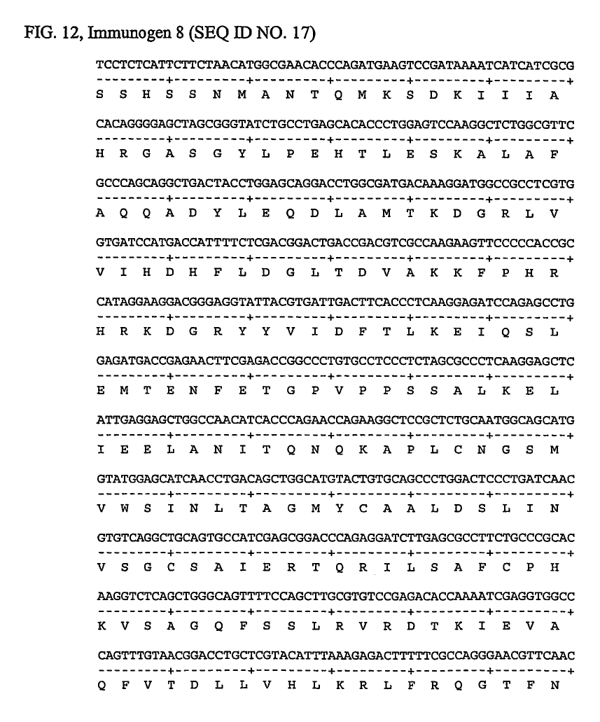

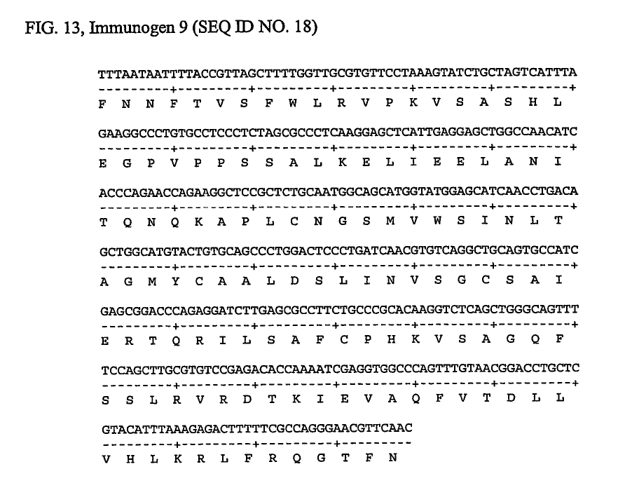

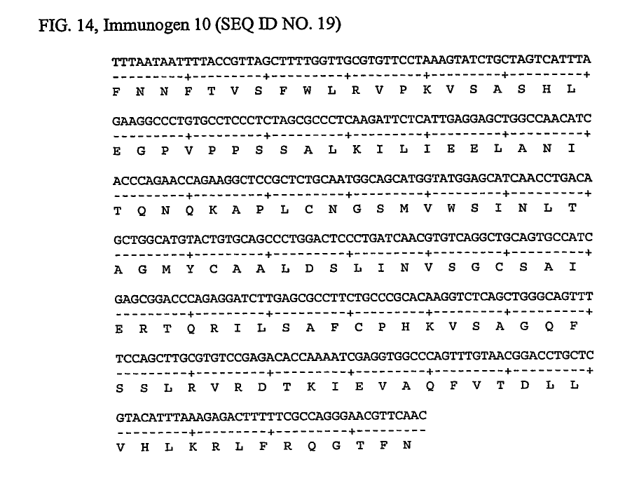

Other preferred immunogens are based on a human chimaeric IL-13 “Immunogen 1” (SEQ ID NO. 10). For example, Immunogen 1 is preferably N-terminally fused to the carrier “CPC” to form Immunogen 7 (SEQ ID NO. 16, see FIG. 11), or N-terminally fused to protein D (the protein D fusion region corresponds to amino acids S20 to T127 inclusive, of H. influenzae protein D sequence (nb, the DNA sequence encoding the protein D is codon optimised) for Immunogen 8 (SEQ ID NO. 17, see FIG. 12); or N-terminally fused to P30 to give Immunogen 9 (SEQ ID NO.18, see FIG. 13). Immunogen 9 preferably further comprises the E121 mutation to abrogate any IL-13 biological activity, to give Immunogen 10 (SEQ ID NO. 19, see FIG. 14).

The protein and DNA sequences shown for Immunogens 1 to 10 are shown without the amino acid or DNA sequence for the signal sequence required to drive secretion of the product from the cell. Preferably, therefore, the sequences further are further provided with a signal sequence. In the context of DNA vaccines it is specifically preferred that the signal sequence is a non-human derived sequence that comprises a T-cell epitope, to further provide T-cell help. None of the disclosed preferred sequences have a stop codon as it may be useful to express them fused to other molecules eg immunoglobulin Fc, 6His to facilitate production or purification.

The numbering system used herein conforms with normal practice in the field of IL-13, in that the G in “GPVPP” is referred to as residue 2, and the remaining amino acids are numbered accordingly.

Methods of Designing a Vaccine

In an important aspect of the present invention, there is provided a method of designing a vaccine for the treatment of an individual suffering from or susceptible to a disease that is susceptible to treatment by neutralisation of the activity of IL-13. Such diseases include COPD, asthma and atopic disorders such as hayfever, contact allergies and atopic dermatitis.

The methods disclosed herein comprise two major steps: 1. Designing a chimaeric IL-13 immunogen, and 2. Associating to the IL-13 immunogen, a source of T-cell epitopes that are foreign with respect to any human self epitope and also foreign with respect to any mammalian IL-13 sequence.

In this context the method comprises:

(a) taking the sequence of human IL-13 and identifying regions that are predicted to form an alpha helical structure, and

(b) mutating the sequence of human IL-13 within these alpha helical regions to substitute amino acids from the human sequence with amino acids that are either a conservative substitution or are found in equivalent positions within the IL-13 sequence of a different species, and

c) attaching or inserting a source of T-cell epitopes that are foreign with respect to any human self epitope and also foreign with respect to any mammalian IL-13 sequence.

As a general principle the object of the method is to design a chimaeric sequence having a maximum sequence diversity between the immunogen and human native IL-13, whilst keeping maximal shape and conformational homology between the two compositions. The chimaeric immunogen achieves this by substituting amino acids found in regions predicted to be masked from the surface. Most preferably the amino acids are substituted with amino acids that are found in equivalent positions within an IL-13 sequence from another mammalian species. In this way, sequence diversity is achieved with minimal alteration to the overall shape/configuration of the immunogen.

Therefore, the preferred methods of designing a chimaeric IL-13 immunogen comprise the following steps:

1. Collect together IL13 sequences from other and align using tool such as Clustal or Pileup,

2. Avoid mutations within positions which are essentially invariant across the collection. Particularly 3PVP (SEQ ID NO. 30), 12ELIEEL (SEQ ID NO. 31), 19NITQ (SEQ ID NO. 32), 28LCN (SEQ ID NO. 33), 32SMVWS (SEQ ID NO. 34), 50SL (SEQ ID NO. 35), 60AI (SEQ ID NO. 36), 64TQ (SEQ ID NO. 37), 87DTKIEVA (SEQ ID NO. 38), 99LL (SEQ ID NO. 39), 106LF (SEQ ID NO. 40),

3. In the remaining sequence, favour mutations that occur in the helical regions

| | |
|---|---|
| (PSTALRELIEELVNIT, | (SEQ ID NO. 41) |
| MYCAALESLI, | (SEQ ID NO. 42) |
| KTQRMLSGF | (SEQ ID NO. 43) |
| or | |
| AQFVKDLLLHLKKLFRE, | (SEQ ID NO. 44) |

4. Regions not specified in 3 or 4 may optionally contain mutations.
5. Mutations are selected by considering either residues which occur in other species IL13 molecules at orthologous positions, or those which are chemically conservative.

Molecular modelling may be used to select particularly favourable substitutions which have a low probability of affecting the overall shape of the molecule by steric clashes etc.

Accordingly there is provided a method for the manufacture of a human chimaeric IL-13 immunogen which has a similar conformational shape to native human IL-13 whilst having sufficient amino acid sequence diversity to enhance its immunogenicity when administered to a human, the method comprising the following steps:

(a) taking the sequence of human IL-13 and performing at least one substitution mutation in at least two of the following alpha helical regions: PSTALRELIEELVNIT (SEQ ID NO. 41), MYCAALESLI (SEQ ID NO. 42), KTQRMLSGF (SEQ ID NO. 43) or AQFVKDLLLHLKKLFRE (SEQ ID NO. 44),

(b) preserving at least six of the following regions of high inter-species conservation 3PVP (SEQ ID NO. 30), 12ELIEEL (SEQ ID NO. 31), 19NITQ (SEQ ID NO. 32), 28LCN (SEQ ID NO. 33), 32SMVWS (SEQ ID NO. 34), 50SL (SEQ ID NO. 35), 60AI (SEQ ID NO. 36), 64TQ (SEQ ID NO. 37), 87DTKIEVA (SEQ ID NO. 38), 99LL (SEQ ID NO. 39), 106LF (SEQ ID NO. 40),

(c) optionally mutating any of the remaining amino acids, and

(d) attaching a source of T-cell epitopes that are foreign with respect to any human self epitope and also foreign with respect to any mammalian IL-13 sequence,

characterised in that any substitution performed in steps a, b or c is a structurally conservative substitution.

In the context of step (a) preferably at least two, more preferably at least three and most preferably all four alpha helical regions comprise at least one substitution mutation. In the context of step (b) preferably at least 7, more preferably at least 8, more preferably at least 9, more preferably at least 10, and most preferably all 11 of the regions are unmutated.

Alternatively there is provided, a method for the manufacture of a human chimaeric IL-13 immunogen which has a similar conformational shape to native human IL-13 whilst having sufficient amino acid sequence diversity to enhance its immunogenicity when administered to a human, the method comprising the following steps:

(a) aligning IL-13 amino acid sequences from different species,

(b) identifying regions of high variability and high conservation,

(c) taking the sequence of human IL-13 and mutating it in the areas of high variability to substitute amino acids from the human sequence with amino acids that are either a conservative substitution or are found in equivalent positions within the IL-13 sequence of a different species, and

(d) attaching a source of T-cell epitopes that are foreign with respect to any human self epitope and also foreign with respect to any mammalian IL-13 sequence,

In a related aspect of the present invention, there is also provided a method for the manufacture of a human chimaeric IL-13 immunogen comprising the following steps:

(a) aligning IL-13 amino acid sequences from different species,

(b) identifying regions of high variability and high conservation,

(c) taking the sequence of human IL-13 and mutating it in the areas of high conservation to substitute amino acids from the human sequence with amino acids that are either a conservative substitution or are found in equivalent positions within the IL-13 sequence of a different species, and

(d) attaching a source of T-cell epitopes that are foreign with respect to any human self epitope and also foreign with respect to any mammalian IL-13 sequence,

In all of these methods, preferably greater than 50% of these substitutions or mutations comprise amino acids taken from equivalent positions within the IL-13 sequence of a non-human. More preferably more than 60, or 70, or 80 percent of the substitutions comprise amino acids taken from equivalent positions within the IL-13 sequence of a non-human mammal. Most preferably, each substitution or mutation comprise amino acids taken from equivalent positions within the IL-13 sequence of a non-human mammal.

Again in the context of the methods for designing chimaeric human immunogens, preferably greater than 50% of these substitutions or mutations occur in regions of human IL-13 which are predicted to be alpha helical in configuration. More preferably more than 60, or 70, or 80 percent of the substitutions or mutations occur in regions of human IL-13 which are predicted to be alpha helical in configuration. Most preferably, each substitution or mutation occurs in regions of human IL-13 which are predicted to be alpha helical in configuration.

Again in the context of the methods of designing chimaeric human immunogens, preferably the immunogen comprises between 2 and 20 substitutions, more preferably between 6 and 15 substitutions, and most preferably 13 substitutions.

Most preferably, in all of these above methods there are substitution mutations in a plurality of sites within the IL-13 sequence, wherein at least two or more of the mutation sites comprise a substitution involving amino acids taken from different non-human mammalian species, more preferably the substitutions involve amino acids taken from 3 or more different non-human mammalian species, and most preferably the substitutions involve amino acids taken from 4 or more different non-human mammalian species.

The present invention also provides an immunogen that is derivable from any of the above methods, which immunogens are immunogenic, when formulated in an appropriate manner for a vaccine, in a human vaccinee.

The successful design of a polypeptide according to the present invention can be verified for example by administering the resulting polypeptide in a self-context in an appropriate vaccination regime, and observing that antibodies capable of binding the protein are induced. This binding may be assessed through use of ELISA techniques employing recombinant or purified native protein, or through bioassays examining the effect of the protein on a sensitive cell or tissue. A particularly favoured assessment is to observe a phenomenon causally related to activity of the protein in the intact host, and to determine whether the presence of antibodies induced by the methods of the invention modulate that phenomenon. Thus a protein of the present invention will be able to raise antibodies to the native antigen in the species from which the native protein is derived.

The most successful of designs will be able to be used in an experiment, such as that described in Example 2 herein, and induce anti-IL-13 neutralising immune responses that exceed ED100 in at least 50% of the vaccinated individuals.

Vaccine Formulations

The vaccine formulations of the present invention may be in the form of a protein based vaccine, most often formulated together with an adjuvant, or alternatively the vaccine may take the form of a DNA or polynucleotide vaccine.

The polypeptide immunogens of the invention may be encoded by polynucleotides of the invention. A person skilled in the art will readily be able to determine the sequence of the polynucleotide which encodes the polypeptide by applying the genetic code. Once the required nucleic acid sequence has been determined, the polynucleotide with the desired sequence can be produced as described in the examples. A skilled person will readily be able to adapt any parameters necessary, such as primers and PCR conditions. It will also be understood by a person skilled in the art that, due to the degeneracy of the genetic code, there is potentially more than one polynucleotide which encodes a polypeptide of the invention. The polynucleotides of the present invention may also comprise a region which encodes a secretion signal peptide.

The polynucleotide of the invention is typically RNA, for example mRNA, or DNA, for example genomic DNA, cDNA or synthetic DNA. Preferably the polynucleotide is DNA. Particularly preferably it is cDNA.

The present invention further provides an expression vector, which is a nucleic acid construct, comprising the polynucleotide of the invention. Additionally, the nucleic acid construct will comprise appropriate initiators, promoters, enhancers and other elements, such as for example, polyadenylation signals, which may be necessary, and which are positioned in the correct orientation, in order to allow for protein expression within a mammalian cell.

The promoter may be a eukaryotic promoter for example a CD68 promoter, Gal1, Gal10, or NMT1 promoter, a prokaryotic promoter for example Tac, Trc, or Lac; or a viral promoter, for example the cytomegalovirus promoter, the SV40 promoter, the polyhedrin promoter, the P10 promoter, or the respiratory syncytial virus LTR promoter. Preferably the promoter is a viral promoter. Particularly preferred is when the promoter is the cytomegalovirus immediate early promoter, optionally comprising exon 1 from the HCMV IE gene.

The transcriptional regulatory elements may comprise enhancers, for example the hepatitis B surface antigen 3′untranslated region, the CMV enhancer; introns, for example the CD68 intron, or the CMV intron A, or regulatory regions, for example the CMV 5′ untranslated region.

The polynucleotide is preferably operably linked to the promoter on the nucleic acid construct such that when the construct is inserted into a mammalian cell, the polynucleotide is expressed to produce a encoded polypeptide.

The nucleic acid construct backbone may be RNA or DNA, for example plasmid DNA, viral DNA, bacterial DNA, bacterial artificial chromosome DNA, yeast artificial chromosome DNA, synthetic DNA It is also possible for the nucleic acid construct to be artificial nucleic acid, for example phosphorothioate RNA or DNA. Preferably the construct is DNA. Particularly preferred is when it is plasmid DNA.

The present invention further provides a host cell comprising an expression vector of the invention. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa, NS0 and COS cells. Preferably the cell line selected will be one which is not only stable, but also allows for mature glycosylation of a polypeptide. Expression may be achieved in transformed oocytes. A polypeptide of the invention may be expressed in cells of a transgenic non-human animal, preferably a mouse or expressed into the milk of larger mammals, such as goats, sheep and cows. A transgenic non-human animal expressing a polypeptide of the invention is included within the scope of the invention. A polypeptide of the invention may also be expressed in Xenopus laevis oocytes.

The present invention also includes pharmaceutical or vaccine compositions, which comprise a therapeutically effective amount of polynucleotide or nucleic acid construct or polypeptide of the invention, optionally in combination with a pharmaceutically acceptable carrier, preferably in combination with a pharmaceutically acceptable excipient such as phosphate buffered saline (PBS), saline, dextrose, water, glycerol, ethanol, liposomes or combinations thereof. The vaccine composition may alternatively comprise a therapeutically effective amount of a nucleic acid construct of the invention, formulated onto metal beads, preferably gold beads. The vaccine composition of the invention may also comprise an adjuvant, such as, for example, in an embodiment, imiquimod, tucaresol or aluminium salts.

Preferably the adjuvant is administered at the same time as the immunogens of the present invention, and in preferred embodiments are formulated together. Such adjuvant agents contemplated by the invention include, but this list is by no means exhaustive and does not preclude other agents: synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules, Th1 inducers such as interferon gamma, IL-2, IL-12, IL-15 and IL-18, Th2 inducers such as IL-4, IL-5, IL-6, IL-10 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

In particular, the adjuvant comprises an immunostimulatory CpG oligonucleotide, such as disclosed in (WO96102555). Typical immunostimulatory oligonucleotides will be between 8-100 bases in length and comprises the general formula X₁CpGX₂ where X₁ and X₂ are nucleotide bases, and the C and G are unmethylated.

The preferred oligonucleotides for use in vaccines of the present invention preferably contain two or more dinucleotide CpG motifs preferably separated by at least three, more preferably at least six or more nucleotides. The oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages. e.g. mixed phosphorothioate/phophodiesters. Other internucleotide bonds which stabilise the oligonucleotide may be used. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

(SEQ ID NO. 62)

OLIGO 1: TCC ATG ACG TTC CTG ACG TT (CpG 1826)

(SEQ ID NO. 63)

OLIGO 2: TCT CCC AGC GTG CGC CAT (CpG 1758)

(SEQ ID NO. 64)

OLIGO 3: ACC GAT GAC GTC GCC GGT GAC GGC ACC ACG

(SEQ ID NO. 65)

OLIGO 4: TCG TCG TTT TGT CGT TTT GTC GTT
(CpG 2006)

(SEQ ID NO. 66)

OLIGO 5: TCC ATG ACG TTC CTG ATG CT (CpG 1668)

Alternative CpG oligonucleotides may comprise the preferred sequences above in that they have inconsequential deletions or additions thereto.

The CpG oligonucleotides utilised in the present invention may be synthesized by any method known in the art (eg EP 468520). Conveniently, such oligonucleotides may be synthesized utilising an automated synthesizer. An adjuvant formulation for use in mice and containing CpG oligonucleotide can be purchased from Qiagen under the trade name “ImmunEasy”. Preferably the adjuvant is one of the CpG's defines as OLIGO's 1, 2, 3, 4 or 5 adsorbed to aluminium hydroxide at an approximate 1:1 ratio weight/weight. OLIGO 4 is preferred for use in humans.

Preferably the CpG is in combination with a saponin, such as QS21, as described in WO 00/62800 and WO 00/09159 the contents of both of which is encorporated herein by reference.

Methods of Treatment

The present invention provides novel treatments for atopic diseases, comprising an immunogen that is capable of generating an immune response in a vaccinee against IL-13. Most notably the present invention provides a method of treating an individual suffering from or being susceptible to COPD, asthma or atopic dermatitis, comprising administering to that individual a vaccine according to the present invention, and thereby raising in that individual a serum neutralising anti-IL-13 immune response and thereby ameliorating or abrogating the symptoms of COPD, asthma or atopic dermatitis.

Also provided by the present invention is the use of the immunogens of the present invention in the manufacture of a medicament for the treatment asthma. Also provided is a method of treatment of asthma comprising the administration to an individual in need thereof of a pharmaceutical composition or vaccine as described herein.

Preferably the pharmaceutical composition is a vaccine that raises an immune response against IL-13. The immune response raised is preferably an antibody response, most preferably an IL-13 neutralising antibody response.

The invention also provides:

an expression vector which comprises a polynucleotide of the invention and which is capable of expressing a polypeptide of the invention;

a host cell comprising an expression vector of the invention;

a method of producing a polypeptide of the invention which method comprises maintaining a host cell of the invention under conditions suitable for obtaining expression of the polypeptide and isolating the said polypeptide:

a vaccine composition comprising a polypeptide or polynucleotide of the invention and a pharmaceutically acceptable carrier.

The methods of treatment of the present invention provide a method of treatment of asthma comprising one or more of the following clinical effects:

1. A reduction in airway hyper-responsiveness (AHR)

2. A reduction in mucus hyper-secretion and goblet cell metaplasia

3. A reduction in sub-epithelial fibrosis of the airways

4. A reduction in eosinophil levels

5. A reduction in the requirement for the use of inhaled corticosteroids (ICS) would also be a feature of successfull treatment using an IL13 autovaccine.

The compositions of the present invention may be used for both prophylaxis and therapy. The present invention provides a polypeptide or a polynucleotide according to the invention for use in medicine. The invention further provides the use of a polypeptide or a polynucleotide of the invention in the manufacture of a medicament for the treatment of allergies, respiratory ailments such as asthma and COPD, helminth-infection related disorders, fibrosis or cirrhosis of the liver.

The present invention also provides a method of vaccinating which comprises administering an effective amount of a vaccine composition of the invention to a patient and provoking an immune response to the vaccine composition.

The present invention also provides vaccine compositions as described herein for use in vaccination of a mammal against IL-13 mediated disorders such as allergies, respiratory ailments, helminth-infection related disorders, fibrosis and cirrhosis of the liver. A vaccine composition capable of directing a neutralising response to IL-13 would therefore constitute a useful therapeutic for the treatment of asthma, particularly allergic asthma, in humans. It would also have application in the treatment of certain helminth infection-related disorders (Brombacher, 2000 Bioessays 22:646-656) and diseases where IL-13 production is implicated in fibrosis (Chiaramonte et al, 1999, J Clin Inv 104:777-785), such as chronic obstructive pulmonary disease (COPD) and cirrhosis of the liver.

The methods of treatment of the present invention provide a method of treatment of atopic dermatitis comprising one or more of the following clinical effects:

1. A reduction in skin irritation

2. A reduction in itching and scratching

3. A reduction in the requirement for conventional treatment.

4. if applicable a reduction in the requirement for the use of topical corticosteroids. An ideal IL13 autovaccine could potentially make ICS steroid treatment redundant, although a reduction in the ‘frequency of use’ or ‘dose required’ of ICS is also envisaged as a valuable outcome.

The present invention also provides methods of treating or preventing IL-13 mediated disease, any symptoms or diseases associated therewith, comprising administering an effective amount of a protein, a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversly the priming may be with the viral vector or with a protein formulation typically a protein formulated in adjuvant and the boost with a DNA vaccine of the present invention.

The present invention provides methods of generating an anti self IL-13 antibody response in a host by the administration of vaccines of the present invention.

The vaccine compositions of the invention may be administered in a variety of manners for example via the mucosal, such as oral and nasal; pulmonary, intramuscular, subcutaneous or intradermal routes. Where the antigen is to be administered as a protein based vaccine, the vaccine will typically be formulated with an adjuvant and may be lyophilised and resuspended in water for injection prior to use. Such compositions may be administered to an individual as an injectable composition, for example as a sterile aqueous dispersion, preferably isotonic. Typically such compositions will be administered intra muscularly, but other routes of administration are possible.

One technique for intradermally administration involves particle bombardment (which is also known as ‘gene gun’ technology and is described in U.S. Pat. No. 5,371,015). Proteins may be formulated with sugars to form small particles or DNA encoding the antigen may be coated on to inert particles (such as gold beads) and are accelerated at speeds sufficient to enable them to penetrate a surface of a recipient (e.g. skin), for example by means of discharge under high pressure from a projecting device. (Particles coated with nucleic acid vaccine constructs of the invention and protein sugar particles are within the scope of the present invention, as are devices loaded with such particles.) Other methods of administering the nucleic acid constructs or compositions containing said constructs directly to a recipient include ultrasound, electrical stimulation, electroporation and microseeding which is described in U.S. Pat. No. 5,697,901.

A nucleic acid construct of the present invention may also be administered by means of specialised delivery vectors useful in gene therapy. Gene therapy approaches are discussed for example by Verme et al, Nature 1997, 389:239-242. Both viral and non-viral systems can be used. Viral based systems include retroviral, lentiviral, adenoviral, adeno-associated viral, herpes viral and vaccinia-viral based systems. Non-viral based systems include direct administration of nucleic acids and liposome-based systems. For example, the vectors may be encapsulated by liposomes or within polylactide co-glycolide (PLG) particles. A nucleic acid construct of the present invention may also be administered by means of transformed host cells. Such cells include cells harvested from a subject. The nucleic acid vaccine construct can be introduced into such cells in vitro and the transformed cells can later be returned to the subject. The nucleic acid construct of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.). Suitable cells include dendritic cells.

The amount of vaccine composition which is delivered will vary significantly, depending upon the species and weight of mammal being immunised, the nature of the disease state being treated/protected against, the vaccination protocol adopted (i.e. single administration versus repeated doses), the route of administration and the potency and dose of the adjuvant compound chosen. Based upon these variables, a medical or veterinary practitioner will readily be able to determine the appropriate dosage level but it may be, for example, when the vaccine is a nucleic acid that the dose will be 0.5-5 μg/kg of the nucleic acid constructs or composition containing them. In particular, the dose will vary depending on the route of administration. For example, when using intradermal administration on gold beads, the total dosage will preferably between 1 μg-10 ng, particularly preferably, the total dosage will be between 10 μg and 1 ng. When the nucleic acid construct is administered directly, the total dosage is generally higher, for example between 50 μg and 1 or more milligram. The above dosages are exempla of the average case.

In a protein vaccine, the amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, most preferably 1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be either a priming or boosting vaccination regime; be administered systemically, for example via the transdermal, subcutaneous or intramuscular routes or applied to a mucosal surface via, for example, intra nasal or oral routes.

There can, of course, be individual instances where higher or lower dosage ranges are merited, and such are within the scope of this invention.

It is possible for the vaccine composition to be administered on a once off basis or to be administered repeatedly, for example, between 1 and 7 times, preferably between 1 and 4 times, at intervals between about 1 day and about 18 months, preferably one month. This may be optionally followed by dosing at regular intervals of between 1 and 12 months for a period up to the remainder of the patient's life. In an embodiment the patient will receive the antigen in different forms in a prime boost regime. Thus for example an antigen will be first administered as a DNA based vaccine and then subsequently administered as a protein adjuvant base formulation. Once again, however, this treatment regime will be significantly varied depending upon the size and species of animal concerned, the amount of nucleic acid vaccine and/or protein composition administered, the route of administration, the potency and dose of any adjuvant compounds used and other factors which would be apparent to a skilled veterinary or medical practitioner.

Throughout this specification the words “comprise” and “include” or variations such as “comprising”, “comprises”, “including”, “includes” etc., are to be construed both inclusively, that is, use of these words will imply the possible inclusion of integers or elements not specifically recited and also in the exclusionary sense in that the words could be read as “consisting”.

As described herein, the present invention relates isolated polypeptides and isolated polynucleotides. In the context of this invention the term “isolated” is intended to convey that the polypeptide or polynucleotide is not in its native state, insofar as it has been purified at least to some extent or has been synthetically produced, for example by recombinant methods, or mechanical synthesis. The term “isolated” therefore includes the possibility of the polypeptides or polynucleotides being in combination with other biological or non-biological material, such as cells, suspensions of cells or cell fragments, proteins, peptides, expression vectors, organic or inorganic solvents, or other materials where appropriate, but excludes the situation where the polynucleotide is in a state as found in nature.

The present invention is exemplified, but not limited to, the following examples.

### EXAMPLE 1

### Methodology

For the methods below the following nomenclature applies:

1. The construct called mouse IL13 (mIL-13) with tetanus toxin p30 epitope inserted into the protein (substituted into the looped region between alpha helices C and D of mouse IL13) is referred to as mIL13p30CD.

2. The construct called mouse IL13 with p30 at the N-terminus, is referred to as mIL13p30.

3. The construct called new chimaeric IL13 design with p30 N-terminus, is referred to as cIL13new.

IL-13 Subcloning/Modifications:

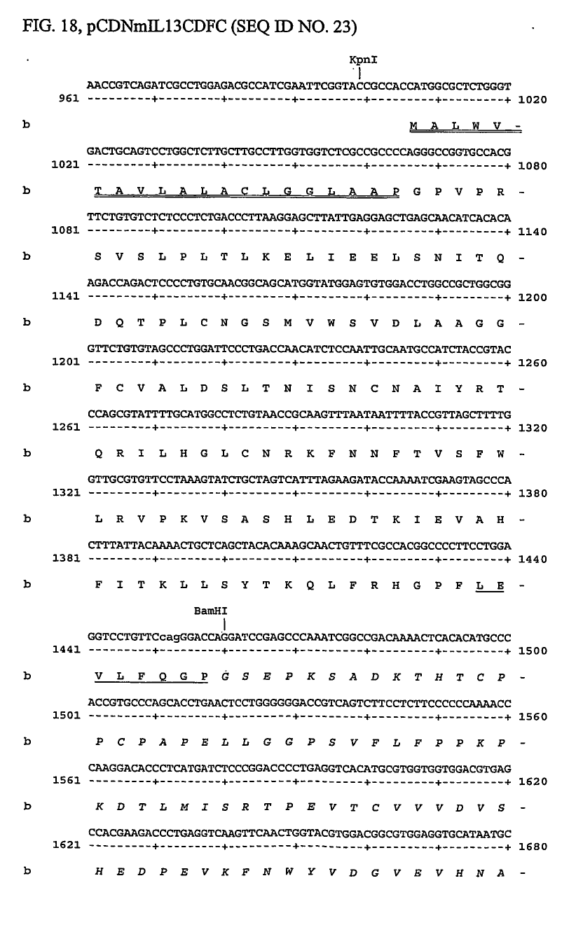

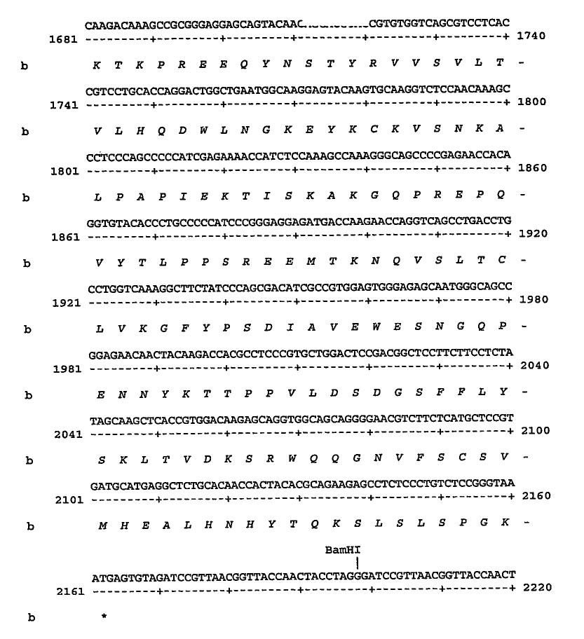

A gene (mIL13CD) encoding mIL-13 containing the p30 epitope from tetanus toxin inserted into the CD loop was prepared synthetically. The synthetic gene contains a 5′ KpnI restriction site and a 3′ BamHI restriction site. This fragment was then subcloned between the Kpn I and Bam HI restriction sites of pCDN which encodes DHFR (Aiyer et al, 1994). The resultant intermediate was subsequently modified by inserting an FC fusion. Site-directed insertional mutagenesis was used to precisely insert human IgG1 FC in frame with the 3′ end coding sequence preceding the stop codon of IL-13 (Geisser et al 2001). This was performed in two steps 1. IgG1 FC was amplified from a cDNA template, pCDN-FC, using the following primer set, (Forward: 5′ . . . CAACTGTTTCGCCACGGCCCCTTCCTGGAGGTCCTGTTCGGTGGACCAGGATCCGAGCCCAAATCGGCCGAC . . . 3′ (SEQ ID NO 67) and Reverse: 5′ . . . CTAGGTAGTTGGTAACCGTTAACGG . . . 3′ (SEQ ID NO. 68)) in a PCR reaction catalyzed by KOD proof-reading polymerase (Novagen). 2. The resultant PCR product was gel purified and 250 ng used as a targeting fragment in a site-directed mutagenesis reaction using the QuickChange kit (Stratagene) with 50 ng mIL-13 CD-pCDN and 2.5 U PfuTurbo. The mutagenesis protocol consisted of 18 Cycles of 30 s at 95° C., 30S at 55° C., and 16 minutes at 68° C. At the end of the mutagenesis protocol, the reaction was digested with 10 U Dpn I to remove the original methylated wild-type template DNA. 1 ul of the final digested reaction was used to transform 100 ul Epicurian chemically competent E. coli cells (Stratagene). Recombinant clones were screened by restriction digestion and positive clones sequence confirmed fully across the FC region using IL-13 forward and pCDN reverse primers. The final plasmid, pCDNmIL13CDFC encodes a C-terminal FC fusion separated by a PreScission protease cleavage site for FC removal. Transcription is under control of the CMV promoter. The complete sequence of the insert is shown in FIG. 18 (SEQ ID NO. 23).

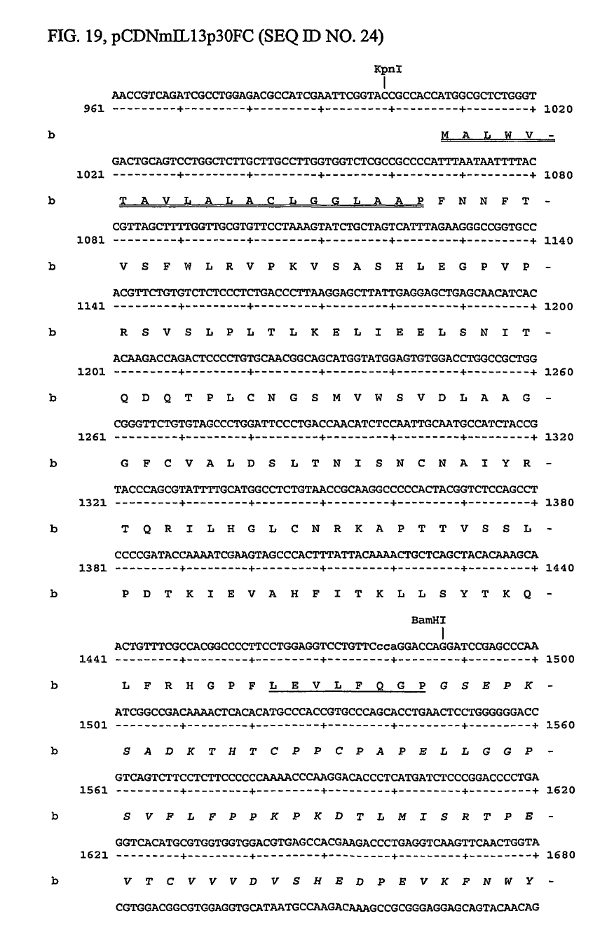

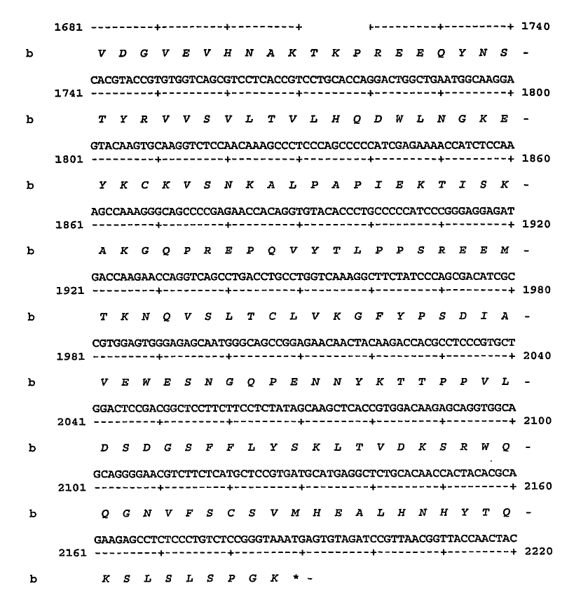

pCDNmIL13p30FC was constructed in exactly the same way as described above for pCDNmIL13CDFC, replacing the mIL13CD synthetic gene with one where the p30 epitope was present at the N terminus of the mature protein instead of being in the CD loop. The same forward and reverse primers were used to generate the targeting fragment for site-directed insertion of the FC region into pCDNmIL13p30. The complete sequence of the insert is shown in FIG. 19 (SEQ ID NO. 24)

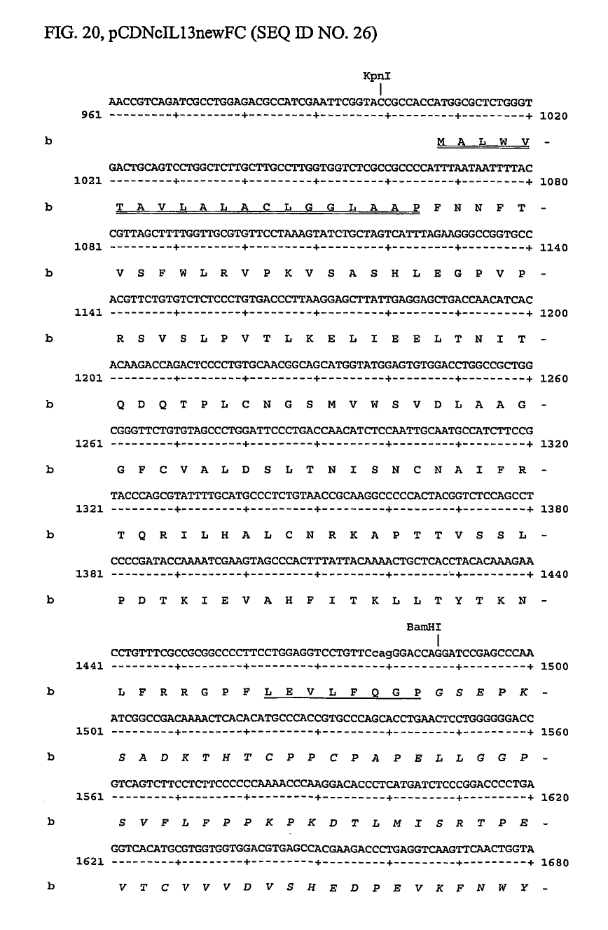

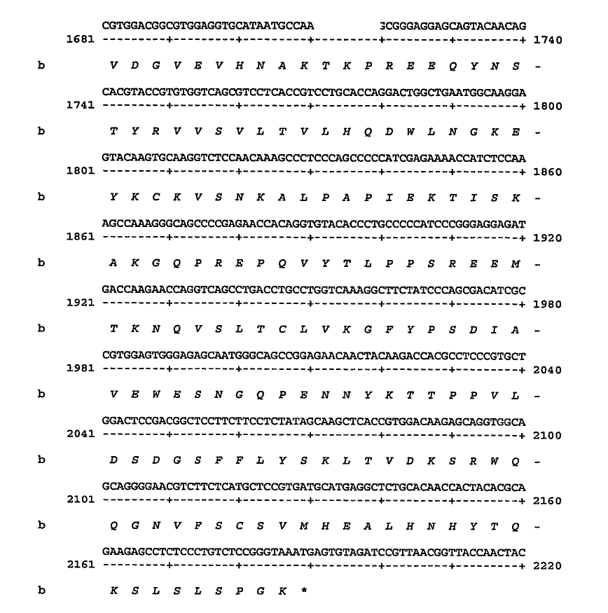

pCDNcIL13newFC was constructed using a synthetic gene encoding the cIL13new molecule and the following forward primer (5′ . . . AACCTGTTTCGCCGCGGCCCCTTCCTGGAGGTCCTGTTCGGTGGACCAGGATCCGAGCCCAAATCGGCCGAC . . . 3′, (SEQ ID NO. 25)) and the same reverse primer described above to generate the targeting fragment for site-directed insertion of the FC region into pCDNcIL13new. The complete sequence of the insert is shown in FIG. 20 (SEQ ID NO. 26)).

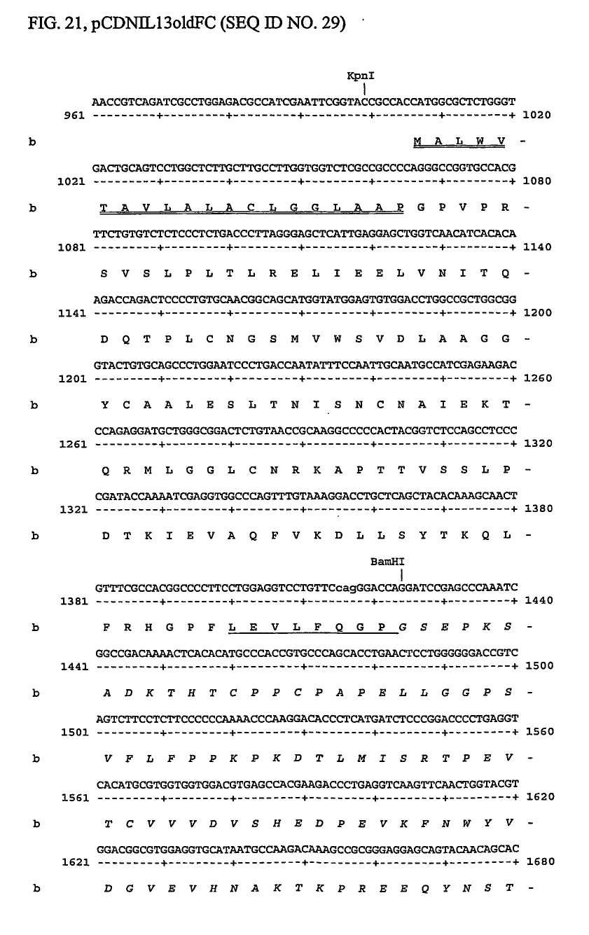

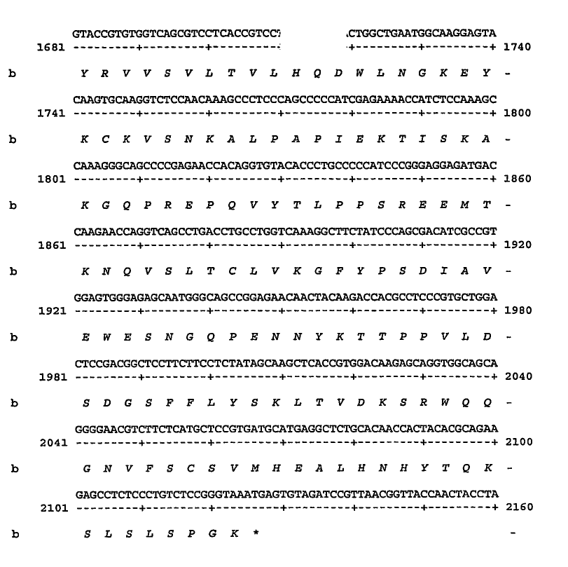

pCDN IL13oldFC was constructed by site-directed replacement of mIL13 CD within pCDNmIL13CDFC with mouse chimeric IL13 (see WO 02/070711). Site-directed replacement was performed as described for site-directed insertion. cIL13 was PCR amplified from 6His-cIL13 using the following primers (Forward: 5′ 5′ . . . GTGTCTCTCCCTCTGACCCTTAGG . . . 3′ (SEQ ID NO. 27) and Reverse: 5′ . . . CAGTTGCTTTGTGTAGCTGAG CAG . . . 3′ (SEQ ID NO. 28) to generate a targeting fragment for replacement into pCDNmIL13. This generates a precise fusion to the IL-13 signal sequence encoded at the 5′ end and the PreScission-FC region encoded at the 3′ end. The complete sequence of the insert is shown in FIG. 21 (SEQ ID NO. 29).

In all of FIGS. 18 to 21, doubly underlined amino acid residues indicate the secretion signal sequence (removed in the course of expression and secretion from the host cell), single underlined residues, the Precission protease site and italicised residues the Fc fusion partner.

Generation of Stable CHO E1A Clones:

Plasmids were stably expressed in a DHFR negative, E1A expressing line (CHO E1A, ACC317). Cells were resuspended at 1×10⁷ cell/ml in cold phosphate buffered sucrose, transferred to a Gene Pulser Cuvette, and electroporated with 15 ug Not I linearized plasmid at 400 volt and 25 uFd in a GenePulser (Biorad). Electroporated cells were plated in a 96 well plate at 2.5×10³ viable cells per well in complete medium containing 1×Nucleosides. After 48 hours the medium was exchanged with fresh medium lacking nucleosides. Cells were subsequently selected over 3-4 weeks in the absence of nucleosides. Positive clones were screened from the 96 well plate by monitoring FC expression from conditioned medium using an FC-electrochemiluminescence detection protocol (Yang, et al., 1994) on an Origen analyzer (IGEN). Positive cell lines were scaled to several litres in complete medium minus nucleosides. Fermentations were carried out at 34° C. for 10-11 days. Conditioned medium was harvested and 0.2 uM sterile filtered in preparation for FC purification.

Purification:

Murine IL13CD/Fc was captured from CHO medium onto ProSep-A High Capacity resin (Bioprocessing Limited). The murine IL13CD/Fc was eluted from the ProSep-A resin with 0.1M Glycine pH=3.0, neutralized with 1M HEPES pH=7.6, and dialyzed against 25 mM sodium phosphate 0.15M sodium chloride pH=7 (Spectra/Por® 7 membrane, MWCO:8000). Overall yield was 644 mg murine IL13CD/Fc from 3.8 liter CHO medium. Other IL13/Fc fusion proteins were prepared similarly.

Before use in vaccination studies, the Fc portions of these molecules were cleaved off using Precission protease and removed. The resulting vaccine preparations comprise essentially those amino acid residues indicated in FIGS. 18 to 21 by plain text (ie neither underlined nor italicised).

### REFERENCES

Aiyer, N, Baker, E, Wu, H-L, Nambi, P, Edwards, R M, Trill, J J, Ellis, C, Bergsma, D J. (1994): Human ATI receptor is a single copy gene: characterization in a stable cell line. Molecular and Cellular Biochemistry 131:75-86.
Geiser, M, Cebe, R, Drewello, D, and Schmitz, R (2001): Integration of PCR Fragments at Any Specific Site within Cloning Vectors without the Use of Restriction Enzymes and DNA Ligase. Biotechniques 31: 88-92.
Yang, H, Leland, J K, Yost, D, Massey, R J (1994): Electrochemiluminescence: A new diagnostic and research tool. Biotechnology, 12:193-194.

### EXAMPLE 2

### Efficacy of an Anti-IL13 Vaccine in a Mouse Asthma Model

The Mouse Asthma Model.

The ovalbumin challenge mouse asthma model is routinely used to assess the efficacy of asthma therapeutic treatments in vivo. Mice are sensitised with 2 intra-peritoneal doses of ovalbumin given 7 days apart, which establishes the sensitivity of the mice to ovalbumin. The asthmatic phenotype can then be generated by giving 3 intra-nasal doses of ovalbumin. Mice subjected to this protocol exhibit a high level of airway hyper-responsiveness to the spasmogen 5HT, inflammation of the lung (most notably an eosinophilia of the lung tissue and broncho-alveolar lavage fluid), and a massive goblet cell metaplasia (and associated mucus hyper-secretion) of the lung airway epithelium. This phenotype mimics that seen in human asthmatics. (Similar mouse asthma models are described in Science 1998 vol 282, pp: 2258-2261 and 2261-2263). This model is also described in WO 02/070711.

Anti-IL13 Vaccine Treatment.

Two anti-IL13 vaccine treatments were assessed for efficacy in the ovalbumin challenge mouse asthma model, in mice that had previously been sensitised to ovalbumin (Sigma UK Ltd, Poole, Dorset). Both are based on the mouse chimeric IL13 molecule disclosed in WO 02/070711, which is expressed and purified as a fusion protein with GST. It is here referred to as gst-cIL13.

1. Vaccine 1=gst-cIL13+‘ImmunEasy’ adjuvant (Qiagen, Cat.No. 303101)

2. Vaccine 2=gst-cIL13+liposomes comprising cholesterol in combination with 10 μg. 3-de-O-acylated monophosphoryl lipid A (3D-MPL) and 10 μg QS21 saponin (see EP0822831B1, SmithKline Beecham Biologicals S.A.)

Negative control vaccine treatment groups were also included.

3. Negative control for vaccine 1=gst+‘ImmunEasy’ adjuvant

4. Negative control for vaccine 2=gst+liposomes comprising cholesterol in combination with 10 μg 3-de-O-acylated monophosphoryl lipid A (3D-MPL) and 10 μg QS21 saponin (see EP0822831B1, SmithKline Beecham Biologicals S.A.). Following sensitisation with ovalbumin mice were immunised with 4 doses of vaccine, each vaccine dose given 4 weeks apart over a 12 week period. Mice were then challenged with ovalbumin and the asthmatic phenotype assessed.

Other Control Treatment Groups in the Efficacy Study.

A. Dexamethasone (Sigma UK Ltd, Poole, Dorset) is a gold-standard steroid treatment routinely used in this mouse asthma model. Mice were given 3 doses of 1.5 mg/kg dexamethasone via the intra-peritoneal route, during ovalbumin challenge.

B. Passively administered anti-mouse IL13 polyclonal antibody (a protein A purfied reagent previously made in-house in rabbits) was given as a positive control treatment in this mouse asthma model. A dose of antibody previously shown to generate fall anti-IL13 driven efficacy in this mouse asthma model was administered during ovalbumin challenge (=3 doses of 0.5 ml of a stock having an endpoint titre of 2×10⁵, for further details see WO 02/070711 A1)

C. The maximum phenotype generated by this model was established in a negative control treatment group using saline (Fresenius Kabi, Warrington, UK). Mice were given 3 doses of saline by the intra-nasal route during ovalbumin challenge. Saline treatment shows no efficacy in this model, therefore the most severe asthmatic phenotype is generated.

D. As a baseline for comparison of the asthma model phenotype to ‘no induced asthmatic phenotype’, one treatment group was only sensitised with ovalbumin, no ovalbumin challenge doses were given. These mice exhibit normal lung physiology.

Serum IL13 Neutralisation Capacity Generated in Mice Immunised with the Anti-IL13 Vaccines, or Passively Administered Anti-IL13 Polyclonal Antibody.

At the end of the mouse asthma model, mice treated with vaccine or passively administered anti-IL13 polyclonal antibody, had serum samples analysed for IL13 neutralisation capacity using the mouse IL13-induced TF-1 cell proliferation assay, as described in WO 02/070711. This analysis yields a neutralisation measure termed ND₅₀, which represents the maximum dilution of mouse serum which is able to reduce by 50% the bioactivity of 5 ng/ml of mouse IL13 in a TF-1 cell proliferation assay.

Our previous data also demonstrated that, using passively administered neutralising anti-IL13 antibodies, maximal efficacy in this murine asthma model is correlated with a serum ND₅₀ value of approximately 1/476. This critical level of neutralisation we term ED₁₀₀ (the effective neutralising dose required to give 100% efficacy), and commonly express serum neutralisation capacities relative to this level. For example, a serum sample which had a ND₅₀ of 1/952 would be said to have a neutralising capacity of 2.0×ED₁₀₀. A sample with a ND₅₀ of 1/238 would have a neutralisation capacity of 0.5×ED₁₀₀.

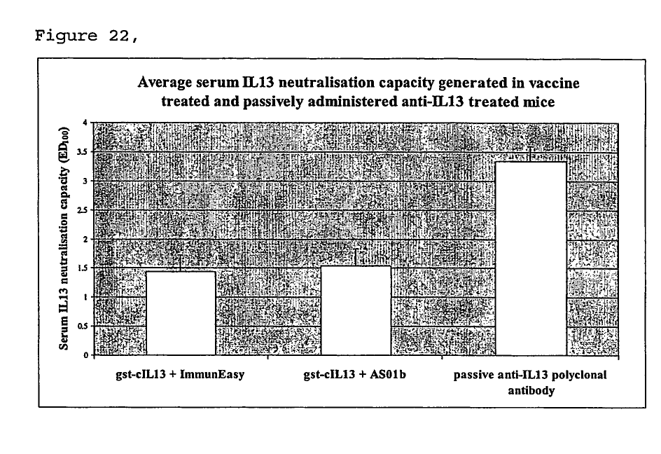

The serum IL13 neutralisation capacity data from this experiment are shown in FIG. 22, and are plotted as a multiples of ED₁₀₀.

All mice that were treated with the chimeric IL13 vaccine or passively adminstered with anti-IL13 polyclonal antibody generated serum neutralisation in excess of 1×ED₁₀₀. Therefore it was predicted that the mice in these treatment groups would receive full anti-IL13 driven benefit in the asthma model.

Airways Hyper-Responsiveness (AHR) Data.

Dose response curves to inhaled spasmogens are used to determine the response of the airways to a bronchoconstrictor stimulus. These curves are comprised of two main components:

1. Hypersensitivity—a leftward shift in the dose response curve (DRC)

2. Hyperreactivity—an increase slope of the DRC and/or a loss in the plateau response

These components together give rise to the general term ‘bronchial or airway hyperresponsiveness’ (BHR or AHR) and this is typically defined as ‘an increase in the ease and degree of airway narrowing in response to bronchoconstrictor stimuli’.

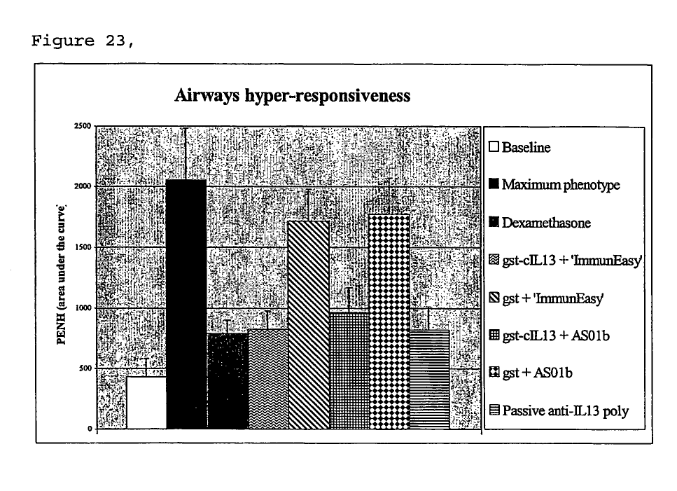

AHR was measured by challenging conscious mice with a dose of 5HT spasmogen, and then measuring the effects on respiratory flow and volume parameters using a whole-body plethysmography apparatus (Buxco, Sharon, Conn.). The preferred readout parameter from this analysis is the measure of enhanced pause (PENH). FIG. 23 illustrates AHR data from this experiment obtained by plotting PENH area under curve values for a 5HT spasmogen concentration of 3 mg/ml. Data points are the means and standard errors for the treatment groups indicated.

Both the vaccine treaments and passively administered anti-IL13 polyclonal antibody were as effective as dexamethasone at reducing the level of AHR. The negative control vaccine treatments did not reduce AHR.

Lung Inflammation Data.

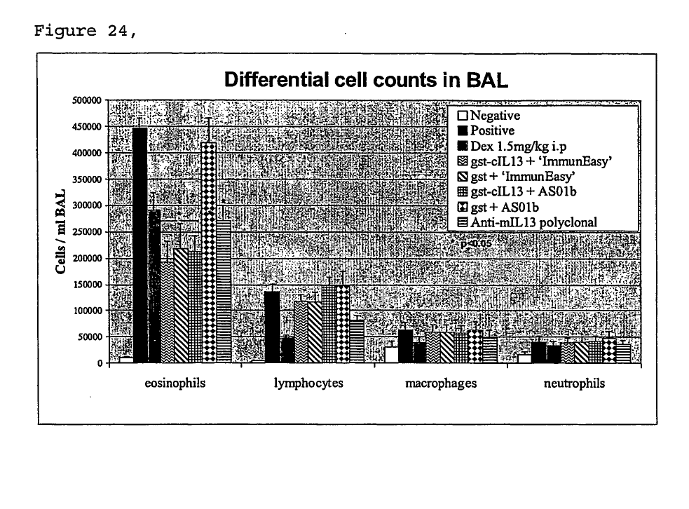

Lung inflammatory cell content was assessed in the broncho-alveolar lavage fluid (BAL). Average numbers of eosinophils, macrophages, lymphocytes and neutrophils were plotted against treatment received (FIG. 24).

Both the vaccine treaments and passively administered anti-IL13 polyclonal antibody were as effective as dexamethasone at reducing the level of eosinophils in the BAL fluid. Interestingly, the negative control treatment gst+‘ImmunEasy’ also appeared to effectively reduce the level of BAL eosinophilia. This is probably due to the activity of the CpG component in the ‘ImmunEasy’ adjuvant which is known to be an immunomodulatory compound with pro-Th1 activity.

Goblet Cell Metaplasia and Mucus Hyper-Secretion Data.

Mucus containing goblet cells are not normally present at significant frequencies in the mouse airway epithelium. Following sensitisation and challenge with ovalubumin in this asthma model, the airway epithelium becomes densely packed with mucus containing goblet cells due to a metaplasia of the epithelial layer.

Following fixation, representative samples of the lungs from each animal were processed for paraffin histology. Sections were cut at 5μ and stained with ABPAS (Alcian blue periodic acid Schiff's reagent, BDH-Merck) with α-amylase (Sigma UK Ltd, Poole, Dorset) pre-digestion for histopathological evaluation of airway goblet cells (preparative histology by Propath UK Ltd, Hereford, UK).

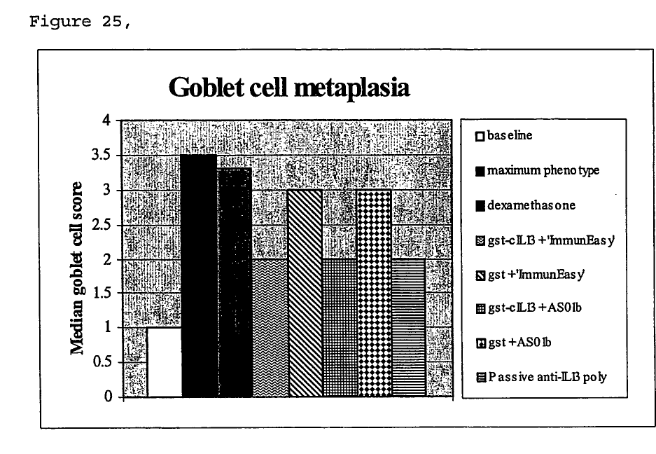

The lung sections stained with ABPAS were scored for goblet cell numbers using the 6-point semi-quantitative scoring system shown below. The results are shown in FIG. 25.

Scoring System for Goblet Cells

| Score | Observation |
|---|---|
| 0 | No goblet cells |
| 1 | Very few goblet cells |
| 2 | Low numbers of goblet cells |
| 3 | Moderate numbers of goblet cells |
| 4 | Heavy numbers of goblet cells |
| 5 | Massive numbers of goblet cells |

Note that the scoring system is not linear, and that the difference between a score of 2 or 3 is highly significant in relation to the number of goblet cells present in the epithelium.

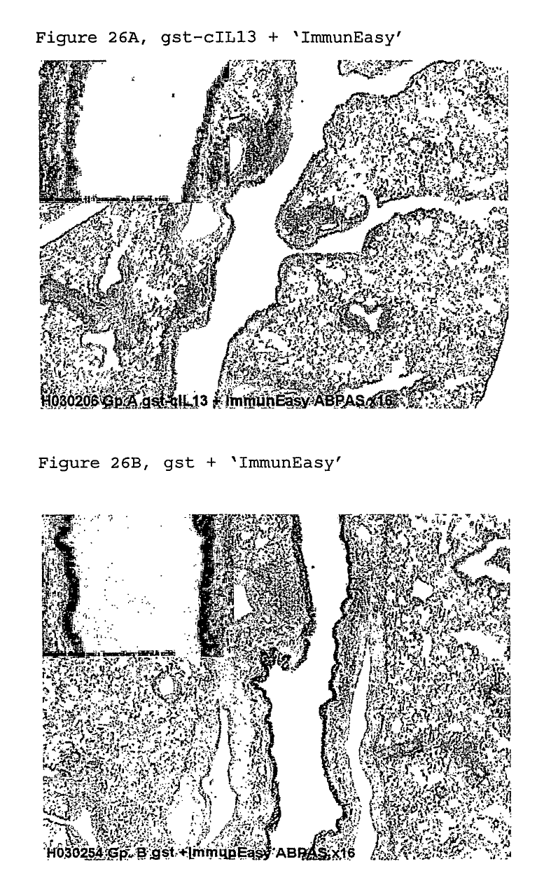

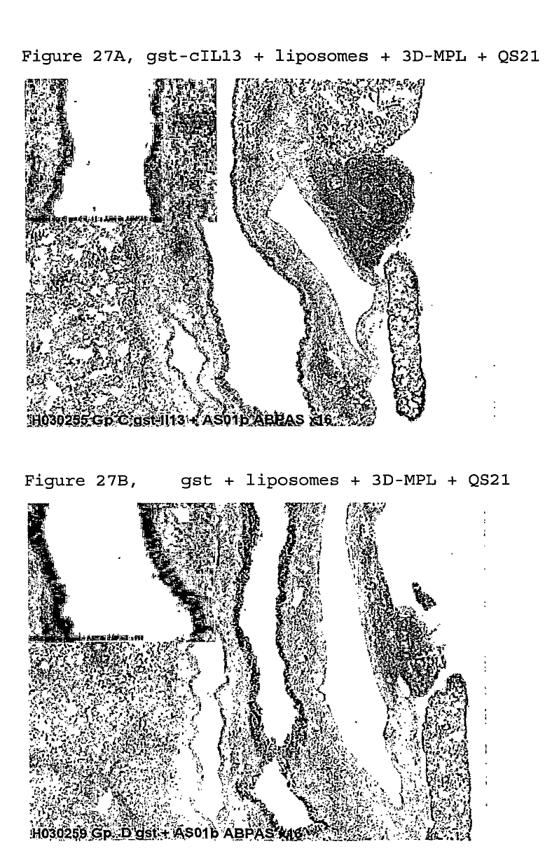

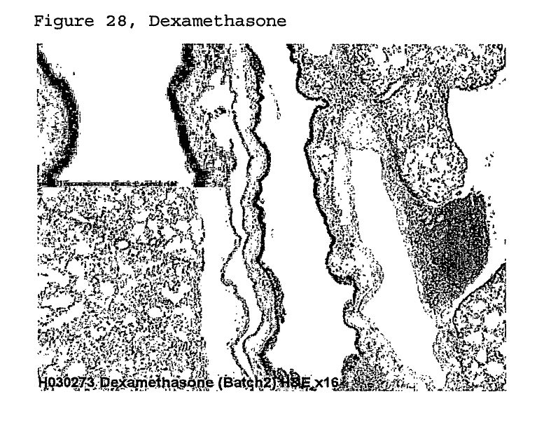

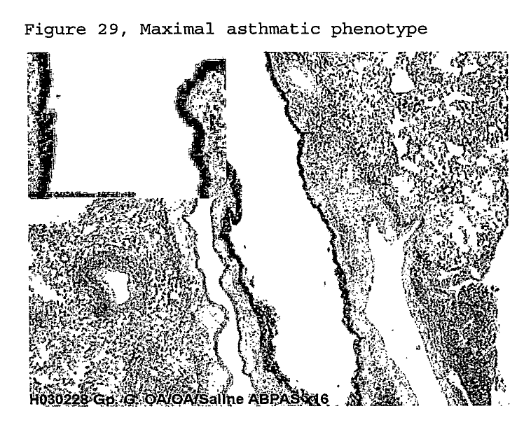

Representative sections for some of the treatment groups are shown in FIG. 26A, gst-cIL13+‘ImmunEasy’; FIG. 26B, gst-‘ImmunEasy’; FIG. 27A, gst-cIL13+Liposomes comprising cholesterol in combination with 10 μg 3-de-O-acylated monophosphoryl lipid A (3D-MPL) and 10 μg QS21 saponin (see EP0822831B1, SmithKline Beecham Biologicals S.A.); FIG. 27B, gst+Liposomes comprising cholesterol in combination with 10 μg 3-de-O-acylated monophosphoryl lipid A (3D-MPL) and 10 μg QS21 saponin (see EP0822831B1, SmithKline Beecham Biologicals S.A.); FIG. 28, dexamethasone; FIG. 29, maximum asthmatic phenotype.

Both the vaccine treaments and passively administered anti-IL13 polyclonal antibody drammatically reduced the numbers of mucus-containing goblet cells in the airway epithelium. The reduction in goblet cell number is highly significant for all anti-IL13 treatments versus the saline (maximum phenotype) treatment group (<0.01). Negative control vaccines had no effect. Dexamethasone treatment had very little effect on goblet cell metaplasia (GCM) in this study.

### SUMMARY

The anti-IL13 vaccine treatments were very effective at abrogating the asthmatic phenotype in the mouse asthma model. Anti-IL13 vaccine was as effective as dexamethasone for treatment of AHR and eosinophilia, and was superior to dexamethasone for treatment of goblet cell metaplasia and mucus hyper-secretion.

### EXAMPLE 3

### Correlation of Goblet Cell Metaplasia with the Level of Serum IL13 Neutralisation Capacity

Some animals immunised with the anti-IL13 vaccines achieved serum IL13 neutralisation levels of less than 1.0×ED₁₀₀. To determine whether these animals were receiving any discernible benefit (keeping in mind that ED₁₀₀ is defined in terms of maximal benefit), they too were challenged with ovalbumin, and the degree of GCM determined. The data below indicates the relationship between goblet cell metaplasia score and level of IL13 neutralisation capacity induced in the serum by the vaccine.

Scoring System for Goblet Cells

| Score | Observation |
|---|---|
| 0 | No goblet cells |
| 1 | Very few goblet cells |
| 2 | Low numbers of goblet cells |
| 3 | Moderate numbers of goblet cells |
| 4 | Large numbers of goblet cells |
| 5 | Massive numbers of goblet cells |

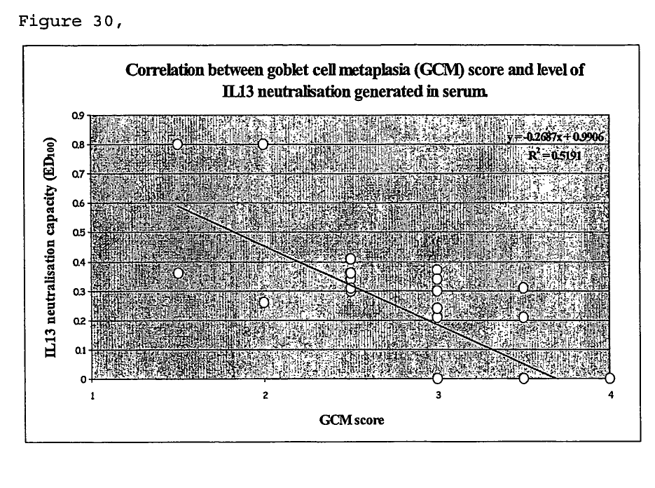

The Goblet cell data is shown in table 1 below and in FIG. 30:

TABLE 1

| | GCM | neut. |
|---|---|---|
| Mouse | score | capacity |
| A1 | 2.5 | 0.41 |
| 2 | 3 | 0.3 |
| 4 | 3.5 | 0.31 |
| 8 | 3.5 | 0.21 |
| 9 | 3.5 | 0 |
| 10 | 2 | 0.6 |
| 11 | 1.5 | 0.36 |
| 12 | 3 | 0.37 |
| 14 | 3 | 0 |
| 15 | 2.5 | 0.3 |
| 16 | 2.5 | 0.34 |
| 18 | 3 | 0 |
| 20 | 3.5 | 0 |
| C30 | 3 | 0 |
| 31 | 3 | 0.21 |
| 33 | 3 | 0 |
| 34 | 4 | 0 |
| 35 | 3.5 | 0 |
| 36 | 3 | 0 |
| 38 | 3 | 0.24 |
| 41 | 2.5 | 0.36 |
| 42 | 3 | 0.34 |
| 43 | 3.5 | 0 |
| 45 | 3 | 0 |
| 46 | 1.5 | 0.8 |
| 47 | 2.5 | 0.31 |
| 48 | 2 | 0.26 |

Only mice that generated serum IL13 neutralisation capacity less than 1×ED₁₀₀ were included in this analysis, because, by definition, animals with a serum IL13 capacity equal to or in excess of 1×ED₁₀₀ achieve a maximal efficacy in respect of suppressing goblet cell metaplasia.

The data indicates that there is a correlation between the level of serum IL13 neutralisation capacity and the severity of goblet cell metaplasia (R²=0.52). The higher the level of IL13 neutralisation, the lower the severity of goblet cell metaplasia.

These data, together with those of Example 3, validate the use of the ED100 measure as a powerful predictor of efficacy of anti-IL13 treatments against the asthmatic phenotype. Any vaccine, antibody, soluble receptor or other IL13 neutralising treatment may be evaluated as follows:
1. Administer the IL13 neutralising treatment to the recipient at the desired dose and frequency.
2. Take a serum sample.
3. Determine the IL13 ND₅₀ of the serum sample by analysing it, and dilutions thereof, in a IL13 bioassay such as the TF1 proliferation assay. The bioassay is chosen such that it is possible to determine the greatest serum dilution which causes a 50% inhibition of the specific effect of 5 ng/ml of mouse IL13. For treatments directed to human IL13, the TF1 bioassay may still be used, but the stimulating cytokine will be human IL13 used at a concentration in the range 3-6 ng/ml.
4. Divide the ND₅₀ value obtained by 1/476 to produce a ED₁₀₀ multiple.
5. If this multiple is 1.0 or greater, the IL13 neutralising treatment is expected to have maximal efficacy on the asthmatic phenotype.
6. If the multiple is considerably less than 1.0, for example 0.2 or less, then no significant efficacy is to be expected.
7. If the multiple lies between these limits, then some efficacy may be seen, but it will not be optimal, indicating that improvements in the treatment will be desirable.
   This process may be used to guide dose selection for maximal efficacy. If, after an initial number of doses of agent, the serum IL13 neutralisation capacity has not reached a level at least equal to 1.0×ED100, then further doses are given to bring the neutralisation capacity up to this level.

### EXAMPLE 4

### Immunogenicity of an Anti-IL13 Protein Vaccine in Combination with Various Adjuvants

Studies to investigate the immunogenicity of a gst-cIL-13 immunogen, with or without the additional promiscuous T-cell epitope P30, in combination with several different adjuvants were performed.

gst-cIL13 Protein Immunogenicity Studies

BalbC mice were immunised with 100 μg gst-cIL13 in adjuvant for the primary immunisation, followed by 50 kg gst-cIL13 in adjuvant for the boost immunisations. Immunisations were administered on a four weekly basis, serum samples taken from mice 2 weeks after each immunisation (to monitor the level of IL13 neutralisation capacity generated by these antibodies in the serum sample). The gst-cIL-13 immunogen was combined with four different adjuvants:

| | |
|---|---|
| Group A | CpG-2006 adsorbed onto aluminium hydroxide |
| Group B | CpG-1826 |
| Group C | CFA prime/IFA boost |
| Group D | aluminium hydroxide |

CpG-2006 and CpG-1826 are oligonucelotides containing unmethylated CG dinucleotides, and well-known in the literature for possessing immunostimulatory activity. CFA/IFA denote complete and incomplete Freunds adjuvant respectively.

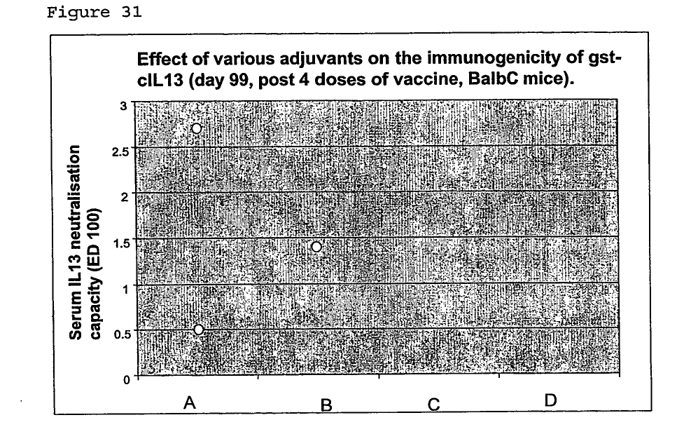

The IL13 neutralisation capacity generated by these antibodies in serum samples was measured in a mouse IL13 bioassay (the TF-1 cell proliferation assay). The table below shows the results (expressed as a multiple of ED₁₀₀) for day 99, post 4 immunisations. The data is also represented graphically in FIG. 31. In this figure, and in the similar figures that follow, each dot indicates a serum IL13 neutralisation measurement for one animal. Animals whose serum neutralising capacity is below the sensitivity threshold of the assay (<0.2×ED₁₀₀) are not plotted.

IL13 neutralisation capacity
expressed as ED₁₀₀

Adjuvant treatment

| BalbC mice | A | B | C | D |
|---|---|---|---|---|
| 1 | <0.2 | <0.2 | <0.2 | <0.2 |
| 2 | 2.7 | <0.2 | <0.2 | <0.2 |
| 3 | 0.5 | <0.2 | <0.2 | <0.2 |
| 4 | <0.2 | 1.4 | <0.2 | <0.2 |
| 5 | <0.2 | <0.2 | <0.2 | <0.2 |

Adjuvant A (CpG (2006) adsorbed onto aluminium hydroxide), in combination with gst-cIL13 protein, was the most effective at generating neutralising anti-IL13 antibody responses. No neutralising anti-IL13 antibody responses were detected for mice treated with gst-cIL13 protein combined with either alum or CFA/IFA adjuvants.

p30-cIL13 Protein.

Study 1

For this study a different form of IL13 vaccine was used. This is another chimeric IL13 molecule which contains the p30 epitope from tetanus toxin at the N terminus. It is encoded by the plasmid pCDNcIL13newFC (FIG. 20), and prepared for vaccine studies as described in Example 1. The fully processed molecule is termed p30-cIL13 in the descriptions below.

Five CD-1 mice were immunised with 40 μg p30-cIL13 in adjuvant for the primary immunisation, followed by 40 μg p30-cIL13 in adjuvant for the boost immunisations. Immunisations were administered on a four weekly basis, serum samples taken from mice 2 weeks after each immunisation (to monitor the level of anti-mouse IL13 antibodies present, and the IL13 neutralisation capacity generated by these antibodies in the serum sample). As a negative control, serum samples were also analysed from three unimmunised CD-1 mice.

| Group | Adjuvant |
|---|---|
| A | Immuneasy ™ (purchased from Qiagen Corp.) |
| B | liposomes comprising cholesterol in combination with |
| | 10 μg 3-de-O-acylated monophosphoryl lipid A (3D-MPL) and |
| | 10 μg QS21 saponin (see EP0822831B1, SmithKline Beecham |
| | Biologicals S.A.). |
| C | No immunisations |

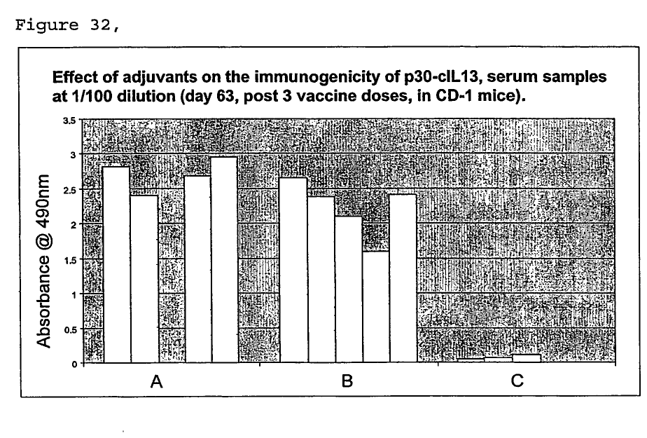

Anti-mouse IL13 antibody levels (in a 1/100 dilution of the serum samples) were measured by ELISA. The table below shows the results (expressed as absorbance at 490 nm) for day 63 post 3 immunisations. The data is also represented graphically in FIG. 32, where each bar represents the data for a single mouse.

| | Absorbance @ 490 nm | | | | |
|---|---|---|---|---|---|
| | Mouse | | | | |
| ELISA data | 1 | 2 | 3 | 4 | 5 |
| A | 2.654 | 2.377 | 2.0995 | 1.5925 | 2.4125 |
| B | 2.81 | 2.398 | n/a | 2.6775 | 2.95 |
| C | 0.049 | 0.0595 | 0.1095 | | |

(n/a = sample not available)

Both adjuvants combined with p30-cIL13 protein were able to raise anti-IL13 antibody responses in CD-1 mice.

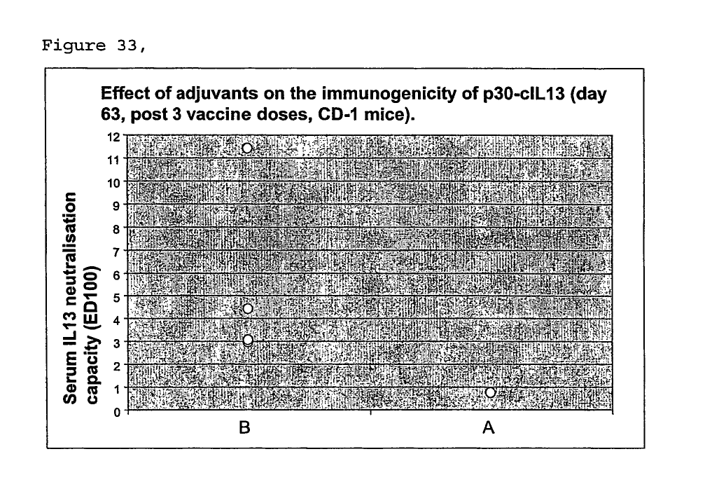

The IL13 neutralisation capacity generated by these antibodies in serum samples was measured in a mouse IL13 bioassay (the TF-1 cell proliferation assay). The table below shows the results (expressed as a multiple of ED₁₀₀) for day 63, post 3 immunisations. The data is also represented graphically in FIG. 33.

IL13 neutralisation capacity
expressed as ED₁₀₀

| CD-1 | | |
|---|---|---|
| mice | A | B |
| 1 | 0.755 | 4.444 |
| 2 | <0.2 | 2.963 |
| 3 | <0.2 | n/a |
| 4 | <0.2 | 11.429 |
| 5 | <0.2 | 3.077 |

Adjuvant B, in combination with p30-cIL13 protein, was the most effective at generating neutralising anti-IL13 antibody responses, 4 out of 5 mice generating potent anti-IL13 neutralising antibody responses in excess of 1×ED₁₀₀. In comparison, only 1 mouse generated neutralising anti-IL13 antibody responses when treated with p30-cIL13 protein combined with ImmunEasy adjuvant (adjuvant A).

Study 2

p30-cIL13 Protein with Oil Emulsion Adjuvant with 3D-MPL and QS21.

Five CD-1 mice were immunised with 40 μg p30-cIL13 in adjuvant for the primary immunisation, followed by 40 μg p30-cIL13 in adjuvant for the boost immunisations. Immunisations were administered on a four weekly basis, serum samples taken from mice 2 weeks after each immunisation (to monitor the level of anti-mouse IL13 antibodies present, and the IL13 neutralisation capacity generated by these antibodies in the serum sample). As a negative control, serum samples were also analysed from three unimmunised CD-1 mice.

| Group | Adjuvant |
|---|---|
| A | ImmunEasy ™ |
| B | oil in water emulsion (oil phase: 1:1 v/v squalene: alpha |
| | tocopherol mix, cholesterol + TWEEN 80 ™ surfactant) + |
| | 10 μg 3D-MPL and 10 μg QS21) (for further |
| | details see WO 99/11241 (described as SB62c')) |
| C | no immunisations |

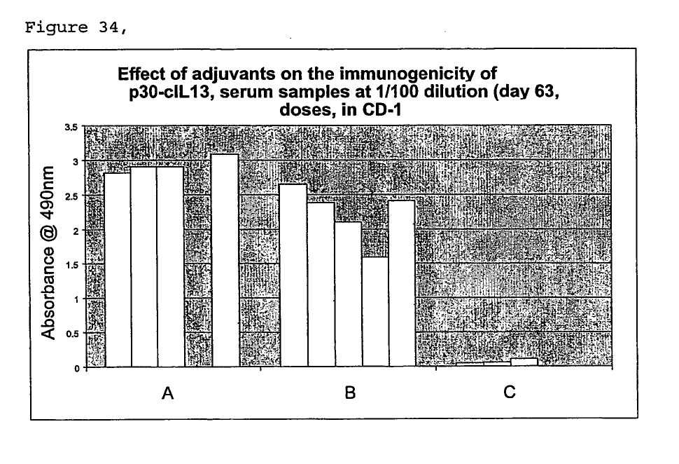

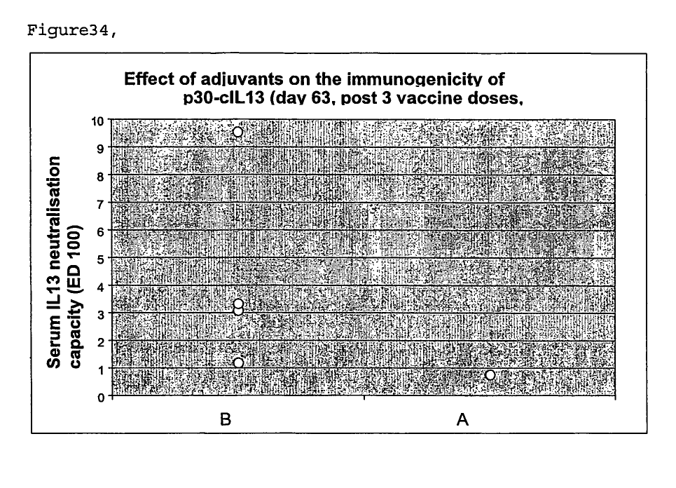

Anti-mouse IL13 antibody levels (in a 1/100 dilution of the serum samples) were measured by ELISA. The table below shows the results (expressed as absorbance at 490 nm) for day 63 post 3 immunisations. The data is also represented graphically in FIG. 34.

| | Absorbance @ 490 nm | | | | |
|---|---|---|---|---|---|
| | Mouse | | | | |
| ELISA data | 1 | 2 | 3 | 4 | 5 |
| A | 2.654 | 2.377 | 2.0995 | 1.5925 | 2.4125 |
| B | 2.8165 | 2.906 | 2.9035 | n/a | 3.081 |
| C | 0.049 | 0.0595 | 0.1095 | | |

Both adjuvants combined with p30-cIL13 protein were able to raise anti-IL13 antibody responses in CD-1 mice.

The IL13 neutralisation capacity generated by these antibodies in serum samples was measured in a mouse IL13 bioassay (the TF-1 cell proliferation assay). The table below shows the results (expressed as a multiple of ED₁₀₀) for day 63, post 3 immunisations. The data is also represented graphically in FIG. 35.

IL13 neutralisation capacity
expressed as ED₁₀₀

| CD-1 | | |
|---|---|---|
| mice | A | B |
| 1 | 0.755 | 3.077 |
| 2 | <0.2 | 9.524 |
| 3 | <0.2 | 3.333 |
| 4 | <0.2 | n/a |
| 5 | <0.2 | 1.176 |

Adjuvant B, in combination with p30-cIL13 protein, was the most effective at generating neutralising anti-IL13 antibody responses, 4 out of 5 mice generating potent anti-IL13 neutralising antibody responses in excess of 1×ED₁₀₀. In comparison, only 1 mouse generated neutralising anti-IL13 antibody responses when treated with p30-cIL13 protein combined with ImmunEasy adjuvant (group A).

Study 3

p30-cIL13 Protein with Oil Emulsion Adjuvant (Without Immunostimulant).

Five CD-1 mice were immunised with 40 μg p30-cIL13 in adjuvant for the primary immunisation, followed by 40 μg p30-cIL13 in adjuvant for the boost immunisations. Immunisations were administered on a four weekly basis, serum samples taken from mice 2 weeks after each immunisation (to monitor the level of anti-mouse IL13 antibodies present, and the IL13 neutralisation capacity generated by these antibodies in the serum sample). As a negative control, serum samples were also analysed from three unimmunised CD-1 mice.

| Group | Adjuvant |
|---|---|
| A | ImmunEasy ™ |
| B | oil in water emulsion (oil phase: 1:1 v/v squalene: alpha |
| | tocopherol mix, cholesterol + TWEEN 80 ™ surfactant) |
| | (for details see WO9517210) |
| C | no immunisations |

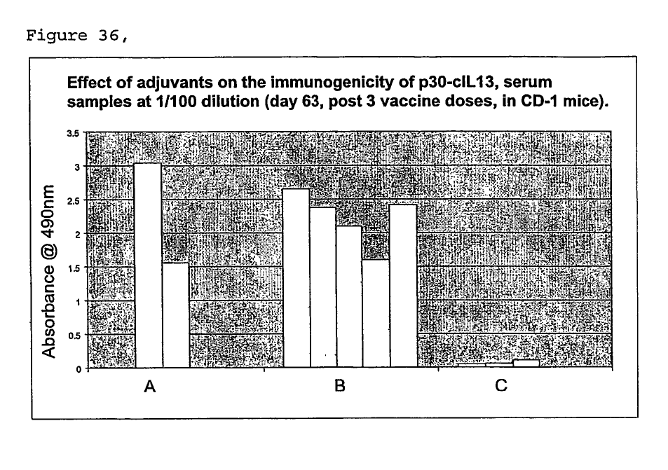

Anti-mouse IL13 antibody levels (in a 1/100 dilution of the serum samples) were measured by ELISA. The table below shows the results (expressed as absorbance at 490 nm) for day 63 post 3 immunisations. The data is also represented graphically in FIG. 36, where each bar represents the data for a single mouse.

| | Absorbance @ 490 nm | | | | |
|---|---|---|---|---|---|
| | Mouse | | | | |
| ELISA data | 1 | 2 | 3 | 4 | 5 |
| A | 2.654 | 2.377 | 2.0995 | 1.5925 | 2.4125 |
| B | n/a | 3.038 | 1.5625 | n/a | n/a |
| C | 0.049 | 0.0595 | 0.1095 | | |

Both adjuvants combined with p30-cIL13 protein were able to raise anti-IL13 antibody responses in CD-1 mice.

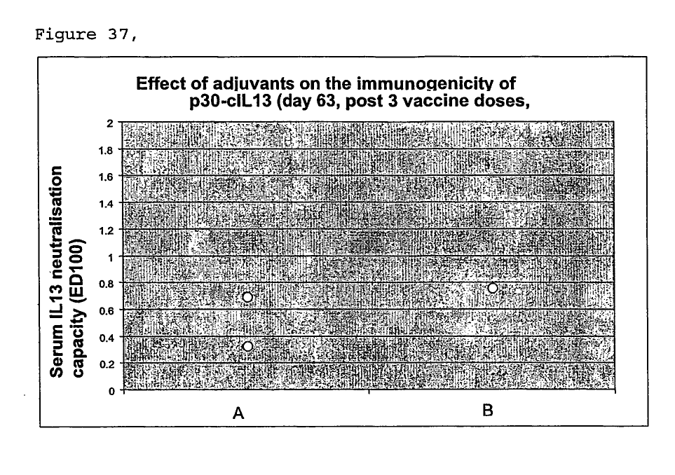

The IL13 neutralisation capacity generated by these antibodies in serum samples was measured in a mouse IL13 bioassay (the TF-1 cell proliferation assay). The table below shows the results (expressed as a a multiple of ED₁₀₀) for day 63, post 3 immunisations. The data is also represented graphically in FIG. 37.

IL13 neutralisation capacity
expressed as ED₁₀₀

| CD-1 | | |
|---|---|---|
| mice | A | B |
| 1 | 0.755 | n/a |
| 2 | <0.2 | 0.32 |
| 3 | <0.2 | 0.69 |
| 4 | <0.2 | n/a |
| 5 | <0.2 | n/a |

Adjuvant B, in combination with p30-cIL13 protein, was the most effective at generating neutralising anti-IL13 antibody responses, 2 out of 5 mice generating anti-IL13 neutralising antibody responses. In comparison, only 1 mouse generated neutralising anti-IL13 antibody responses when treated with p30-cIL13 protein combined with ImmunEasy adjuvant (adjuvant A).

### SUMMARY

The ability of the P30 immunogens to augment the immune response in the outbred CD-1 mouse strain is significant in that is suggests that the use of this immunogen is not limited to a single immunological background, and the advantageous effects of P30 should also be obtained in an outbred human clinical setting.

1. An immunogenic composition comprising an IL-13 element that drives an immune response that recognizes human IL-13 and at least one foreign T-cell epitope.

2. An immunogenic composition as claimed in claim 1, wherein the T-cell epitope is foreign with respect to human self-proteins and IL-13 sequence.

3. An immunogenic composition as claimed in claim 1, wherein the T-cell epitope is a short peptide sequence added to the IL-13 sequence.

4. An immunogenic composition as claimed in claim 3 wherein the carrier protein is selected from the group of: Haemophilus influenzae Protein D and CPC (clyta-P2-clyta).

5. (canceled)

6. An immunogenic composition as claimed in claim 3, wherein at least one short T-cell epitope is added to the IL-13 sequence by an event selected from the group of: an addition and a substitution.

7. An immunogenic composition as claimed in claim 6 wherein the short T-cell epitope is a promiscuous epitope.

8. An immunogenic composition as claimed in claim 7 wherein the promiscuous epitope is selected from the group of: P2 and P30 from tetanus toxoid.

9. An immunogenic composition as claimed in claim 1, wherein the IL-13 element comprises the entire human IL-13 sequence.

10. An immunogenic composition as claimed in claim 9 wherein the IL-13 element is in mutated form.

11. An immunogenic composition as claimed in claim 10, wherein the mutated IL-13 is in the form of a chimaeric IL-13 formed by substituting amino acids with amino acids that are found in equivalent positions within an IL-13 sequence from another mammalian species.

12. An immunogenic composition as claimed in claim 11, wherein the substitutions occur in areas that are associated with alpha helical regions.

13. An immunogenic composition as claimed in claim 11 wherein the substitutions involve amino acids taken from more than one different non-human mammalian species.

14. An immunogenic composition as claimed in claim 1 wherein the IL-13 element is human chimaeric IL-13 sequence having a similar conformational shape to native human IL-13 and sufficient amino acid sequence diversity to enhance its immunogenicity when administered to a human, wherein the human chimaeric IL-13 sequence has the sequence of human IL-13 comprising:
(a) substitution mutations in at least two of the following alpha helical regions selected from the group of: PSTALRELIEELVNIT, MYCAALESLI, KTQRMLSGF and AQFVKDLLLHLKKLFRE;
(b) comprises in unmutated form at least six regions of high inter-species conservation selected from the group of: 3PVP, 12ELIEEL, 19NITQ, 28LCN, 32SMVWS, 50SL, 60AI, 64TQ, 87DTKIEVA, 99LL, and 106LF; and
(c) optionally comprises a mutation in any of the remaining amino acids, wherein any substitution performed in steps a, b or c is a structurally conservative substitution.

15. An immunogenic composition as claimed in claim 14, wherein greater than 50% of these substitutions or mutations comprise amino acids taken from equivalent positions within the IL-13 sequence of a non human.

16. An immunogenic composition as claimed in claim 14, wherein greater than 50% of these substitutions or mutations occur in regions of human IL-13 which are predicted to be alpha helical in configuration.

17. An immunogenic composition as claimed in claim 14, wherein the human chimaeric IL-13 sequence has the sequence of human IL-13 comprising between 2 and 20 substitutions.

18. An immunogenic composition as claimed in claim 1 wherein the IL-13 element is based on a non-human IL-13 sequence wherein the non-human surface exposed regions are substituted for the equivalent human sequences.

19. An immunogenic composition as claimed in claim 14, wherein the amino acid sequence of human IL-13 comprises conservative substitutions in at least six of the following positions selected from the group of: 8T, 11R, 18V, 49E, 62K, 66M, 69G, 84H, 97K, 101L, 105K, 109E, and 111R.

20. An immunogenic composition as claimed in claim 19 comprising at least six of the following substitutions selected from the group of: 8T to S, 11R to K, 18V to A, 49E to D, 62K to R, 66M to I, 69G to A, 84H to R, 97K to T, 101L to V, 105K to R, 109E to Q, and 111R to T.

21. An immunogenic composition as claimed in claim 1, wherein the IL-13 element is selected from the group of: Immunogen 1, Immunogen 11, Immunogen 12 and Immunogen 13.

22. An immunogenic composition as claimed in claim 1, selected from the group of: Immunogen 2, Immunogen 3, Immunogen 7, Immunogen 8, Immunogen 9 and Immunogen 10.

23. An immunogenic composition as claimed in claim 1 further comprising a mutation in the human IL-13 element that abolishes the human IL-13 biological activity and is selected from the group of: E12 to I, S, or Y; E12 to K; R65 to D; S68 to D; and R108 to D.

24. A method of designing an immunogenic composition as claimed in claim 1 comprising:
(a) identifying regions in human IL-13 (SEQ ID NO. 1) that are predicted to form an alpha helical structure;
(b) mutating the sequence of human IL-13 within these alpha helical regions to substitute amino acids from the human sequence with amino acids that are either a conservative substitution or are found in equivalent positions within the IL-13 sequence of a different species; and
(c) attaching or inserting a source of T-cell epitopes that are foreign with respect to any human self epitope and also foreign with respect to any mammalian IL-13 sequence.

25. A method for the manufacture of a human chimaeric IL-13 immunogen which has a similar conformational shape to native human IL-13 and sufficient amino acid sequence diversity to enhance its immunogenicity when administered to a human comprising the following steps:
(a) performing at least one substitution mutation in human IL-13 (SEQ ID NO. 1) in at least two of the following alpha helical regions selected from the group of: PSTALRELIEELVNIT, MYCAALESLI, KTQRMLSGF and AQFVKDLLLHLKKLFRE;
(b) preserving at least six regions of high inter-species conservation selected from the group of: 3PVP, 12ELIEEL, 19NITQ, 28LCN, 32SMVWS, 50SL, 60AI, 64TQ, 87DTKIEVA, 99LL, and 106LF;
(c) optionally mutating any of the remaining amino acids; and
(d) attaching a source of T-cell epitopes that are foreign with respect to any human self epitope and also foreign with respect to any mammalian IL-13 sequence, wherein any substitution performed in steps a, b or c is a structurally conservative substitution.

26. A method for the manufacture of a human chimaeric IL-13 immunogen as claimed in claim 25, wherein all four alpha helical regions comprise at least one substitution mutation.

27. A method for the manufacture of a human chimaeric IL-13 immunogen as claimed in claim 25, wherein there are no mutations at any region of high inter-species conservation.

28. A method for the manufacture of a human chimaeric IL-13 immunogen which has a similar conformational shape to native human IL-13 and sufficient amino acid sequence diversity to enhance its immunogenicity when administered to a human, the method comprising the following steps:
(a) aligning IL-13 amino acid sequences from different species;
(b) identifying regions of high variability and high conservation;
(c) mutating human IL-13 (SEQ ID NO. 1) in the areas of high variability to substitute amino acids from the human sequence with amino acids that are either a conservative substitution or are found in equivalent positions within the IL-13 sequence of a different species; and
(d) attaching a source of T-cell epitopes that are foreign with respect to any human self epitope and also foreign with respect to any mammalian IL-13 sequence.

29. A method for the manufacture of a human chimaeric IL-13 immunogen as claimed in claim 24, wherein all greater than 50% of these substitutions or mutations comprise amino acids taken from equivalent positions within the IL-13 sequence of a non-human species.

30. A method for the manufacture of a human chimaeric IL-13 immunogen as claimed in claim 24, wherein greater than 50% of these substitutions or mutations occur in regions of human IL-13 which are predicted to be alpha helical in configuration.

31. A method for the manufacture of a human chimaeric IL-13 immunogen as claimed in claim 24, wherein substitutions or mutations comprise amino acids taken from equivalent positions within at least two non-human IL-13 sequences.

32. A method for the manufacture of a human chimaeric IL-13 immunogen as claimed in claim 24, wherein the immunogen comprises between 6 and 20 substitutions, and most preferably between 6 and 10 substitutions.

33. An immunogen derived from the method claimed in claim 24, wherein the immunogens are immunogenic, when formulated in an appropriate manner for a vaccine, in a human vaccinee.

34. A vaccine comprising the IL13 element as claimed in claim 1.

35. A polynucleotide vaccine comprising a polynucleotide that encodes a IL13 element as claimed in claim 1.

36. A method of treating an individual suffering from or being susceptible to CPD, asthma or atopic dermatitis, comprising administering to said individual a vaccine as claimed in claim 34, and thereby raising in that individual a serum neutralizing anti-IL-13 immune response and thereby ameliorating or abrogating the symptoms of COPD, asthma or atopic dermatitis.

37-38. (canceled)

39. An immunogenic composition as claimed in claim 1, wherein the T-cell epitope comprises a carrier protein.

40. An immunogenic composition as claimed in claim 39, wherein the carrier protein and IL-13 element form a fusion protein.

41. An immunogenic composition as claimed in claim 3, wherein at least one short T-cell epitope is added to the IL-13 sequence at a terminal end of the IL-13 sequence by means selected from the group of: synthetic, recombinant and molecular biology.

42. An immunogenic composition as claimed in claim 1, wherein the IL-13 element comprises functional equivalent fragments of the human IL-13 sequence.
